(19) United States
(12) Patent Application Publication
(10) Pub. No.: US 2014/0128272 A1
(43) Pub. Date: May 8, 2014

(54) Method for Inducing Dormancy of Cancer Tissue-Derived Cell Mass and Method for Evaluating Treating Means with the Use of Cancer-Tissue-Derived Cell Mass

(71) Applicants: Osaka Prefectural Hospital Organization, Osaka (JP); Renaissance Energy Investment Co., Ltd., Hyogo (JP)

(72) Inventor: Masahiro Inoue, Osaka (JP)

(21) Appl. No.: 13/768,964

(22) Filed: Feb. 15, 2013

Related U.S. Application Data
(60) Provisional application No. 61/724,131, filed on Nov. 8, 2012.

(51) Int. Cl. G01N 33/50

(52) U.S. Cl. CPC G01N 33/5044
USPC 506/9; 435/29; 435/6.13; 435/325; 435/7.23; 435/6.11; 556/40; 544/309; 546/48

(57) ABSTRACT

Provided are a method for retaining in a dormant state a cancer tissue-derived cell mass that can reflect accurately the in vivo behavior of cancer cells, and an evaluation method for examining the sensitivity to various treatments including a drug sensitivity test by using a cancer tissue-derived cell mass in such a dormant state. The cancer tissue-derived cell mass is prepared from an individual. Such a cancer tissue-derived cell mass is cultured in vitro under the conditions of hypoxia and low levels of growth factors. Furthermore, a treatment with a drug, etc. is applied in vitro to the cancer tissue-derived cell mass in the dormant state so that evaluation is achieved by examination of its proliferation state, determination of life and death, and analysis of signals.

### BACKGROUND OF THE INVENTION

1. Field of the Invention

The present invention relates to a method for inducing a dormant state of cancer tissue-derived cell mass, a cancer tissue-derived cell mass in a dormant state, and a method for evaluating various means of treating a cancer tissue-derived cell mass in a dormant state. More particularly, the present invention relates to a method for evaluating the effects of various treatment means such as drugs, etc., which method includes the steps of culturing a cancer tissue-derived cell mass, which is able to reconstitute a cancer in vitro and retain a proliferation ability, under the conditions of hypoxia, etc. so that metabolically inert conditions are maintained without substantial cell death and without substantial cell growth and with reduction in glucose consumption as well as in oxygen consumption; reproducing a state in vitro where the proliferation ability and metabolism can be recovered by culturing the cancer tissue-derived cell mass thereafter in a medium containing oxygen and growth factors; and using such a state for various evaluations described above.

2. Background of Invention

In recent years, therapeutic results of early-stage cancers have been drastically improved as a result of various studies that have been repeated to overcome cancers. However, it is still difficult to treat advanced-stage cancers, and cancers have continued to occupy the first place of the Japanese cause of death. According to vital statistics of 2007 by the Ministry of Health, Labor and Welfare, 340,000 people or more died of cancers a year.

For cancer research so far, especially when examining its behavior in vitro, experiments using a cancer cell line that has been subcultured and established under optimized culture conditions are the mainstream. For a realization of diagnosis or treatment according to individual cancer patients, primary culture of cancer cells is promising, and its research has been attempted. For example, a CD-DST method (Collagen gel droplet embedded drug sensitivity test) using a primary culture cell has been developed. This in vitro test method is a drug sensitivity test by embedding a tissue or a cell isolated from a patient into a collagen gel droplet, and examining the sensitivity by the combination of a three-dimensional culture and an image colorimetric quantification (for example, Takamura Y. et al., (2002) Prediction of chemotherapeutic response by collagen gel droplet embedded culture-drug sensitivity test in human breast cancers, Int. J. Cancer, 98, 450-455).

In addition, a method for culturing a cancer tissue-derived cell mass in vitro has been established by the present inventor(s) (WO 2010/101119, etc.).

### SUMMARY OF THE INVENTION

Cancer cells that are actively growing and cancer cells that are inactive in the growth and metabolism are copresent in the cancer tissue. In the cancer therapy in the past, a main target has been directed to cancer cells in a growing state. Therefore, cancer cells in the so-called dormant state of which growth and metabolism are inactive, are resistant to many existing chemotherapies, as well as to radiation therapies. The dormant state of such cancer cells is considered to be a reversible state that may be released, and this phenomenon is thought to cause the recurrence of cancers after therapy.

One of the objectives of the present invention is to provide a method for mimicking the so-called dormant state in vitro among the behaviors of cancer cells in the living body with use of a cancer tissue-derived cell mass and evaluating the effects of various treatment (therapy) means including, in such a dormant state, drug administration, radiation exposure, thermotherapy, photochemotherapy, immunotherapy, and gene introduction.

Furthermore, another objective of the present invention is to provide a method for evaluating the effects of various treatment means to confer induction and release of dormant state by using a cancer tissue-derived cell mass, such as drug administration, radiation exposure, thermotherapy, photochemotherapy, immunotherapy, and gene introduction.

Focusing on the situation in which cancer cells in a dormant state do not respond to treatments such as existing drugs and radiation exposure, the present inventor(s) intended to evaluate the effects of various treatment means such as drugs, etc. from the viewpoint that was different from conventional viewpoints, extensively studied on the dormant state of cancer cells as a research material to solve the above problems, and found that the in vivo dormant state of cancer cells was effectively reconstructed in vitro using a cancer tissue-derived cell mass, thereby to complete the present invention.

That is, the present invention relates to a method of processing a cancer tissue-derived cell mass, which method includes a step of culturing in vitro the cancer tissue-derived cell mass from a patient under the conditions of hypoxia and low levels of growth factors, the cancer tissue-derived cell mass being a substantially spherical shape or spheroidal form and not containing substantially cells other than cancer cells.

In addition, the present invention relates to a method for evaluating the effect of a treatment for a cancer tissue-derived cell mass, which method includes a step of applying a treatment to the cancer tissue-derived cell mass in a dormant state in vitro with no substantial growth and no substantial cell death as well as with reduction in glucose consumption and oxygen consumption, wherein its proliferation ability is inherently retained, the cancer tissue-derived cell mass being a substantially spherical shape or spheroidal form and not containing substantially cells other than cancer cells.

In the method for evaluating the effect of a treatment, such treatment maybe selected from the group consisting of drug administration, radiation exposure, thermotherapy, immunotherapy, photochemotherapy, and gene introduction.

The method for evaluating the effect of a treatment may be a method that includes a step of detecting proliferation state of the cancer tissue-derived cell mass, determining life and death of the cancer tissue-derived cell mass, or detecting change of intracellular signal transduction of the cancer tissue-derived cell mass.

The present invention also relates to a method for evaluating the effect of a treatment for a cancer tissue-derived cell mass, which method includes a step of culturing in vitro the cancer tissue-derived cell mass, while applying various treatments, from a patient, and then culturing it under the conditions of hypoxia and low levels of growth factors, the cancer tissue-derived cell mass being a substantially spherical shape or spheroidal form and not containing substantially cells other than cancer cells.

The above treatment may be selected from the group consisting of drug administration, radiation exposure, thermotherapy, immunotherapy, photochemotherapy, and gene introduction.

The method for evaluating the effect of a treatment maybe a method that includes a step of detecting proliferation state of the cancer tissue-derived cell mass, determining life and death of the cancer tissue-derived cell mass, or detecting change of intracellular signal transduction of the cancer tissue-derived cell mass.

The present invention also relates to a method for evaluating the effect of a treatments for a cancer tissue-derived cell mass from a patient, which method includes a step of culturing the cancer tissue-derived cell mass in vitro under the conditions of hypoxia and low levels of growth factors, a step of applying a treatment to the cancer tissue-derived cell mass, and a step of culturing the cancer tissue-derived cell mass under the conditions of at least 1 ng/ml to 200 ng/ml of a growth factor, the cancer tissue-derived cell mass being a substantially spherical shape or spheroidal form and not containing substantially cells other than cancer cells.

The treatment may be selected from the group consisting of drug administration, radiation exposure, thermotherapy, immunotherapy, photochemotherapy, and gene introduction.

The method for evaluating the effect of treatments may be a method that includes a step of detecting proliferation state of the cancer tissue-derived cell mass, determining life and death of the cancer tissue-derived cell mass, or detecting change of intracellular signal transduction of the cancer tissue-derived cell mass.

The evaluation method described above may include a step of obtaining genetic information by evaluating the gene of the cancer tissue-derived cell mass in advance and of correlating the genetic information with the treatment.

The present invention relates to a method for screening a drug, which method includes a step of treating a cancer tissue-derived cell mass in a dormant state with a drug candidate compound in vitro, which cancer tissue-derived cell mass in a dormant state is with no substantial growth and no substantial cell death as well as with reduction in glucose consumption and oxygen consumption, wherein its proliferation ability is inherently retained, the cancer tissue-derived cell mass being a substantially spherical shape or spheroidal form and not containing substantially cells other than cancer cells.

The method for screening a drug includes a step of detecting proliferation state of the cancer tissue-derived cell mass, determining life and death of the cancer tissue-derived cell mass, or detecting change of intracellular signal transduction of the cancer tissue-derived cell mass.

The present invention also relates to a method for screening a drug, which method includes a step of culturing in vitro a cancer tissue-derived cell mass from a patient in the presence of a drug candidate compound, and then culturing it under the conditions of hypoxia and low levels of growth factors, the cancer tissue-derived cell mass being a substantially spherical shape or spheroidal form and not containing substantially cells other than cancer cells.

The method for screening a drug may include a step of detecting proliferation state of the cancer tissue-derived cell mass, determining life and death of the cancer tissue-derived cell mass, or detecting change of intracellular signal transduction of the cancer tissue-derived cell mass.

The present invention also relates to a method for screening a drug, which method includes steps of culturing a cancer tissue-derived cell mass from a patient in vitro under the conditions of hypoxia and low levels of growth factors, applying a drug candidate compound to the cancer tissue-derived cell mass, and culturing the cancer tissue-derived cell mass under the conditions of at least 1 ng/ml to 200 ng/ml of a growth factor, the cancer tissue-derived cell mass being a substantially spherical shape or spheroidal form and not containing substantially cells other than cancer cells.

The method for screening a drug may include a step of detecting proliferation state of the cancer tissue-derived cell mass, determining life and death of the cancer tissue-derived cell mass, or detecting change of intracellular signal transduction of the cancer tissue-derived cell mass.

The screening method described above may include a step of obtaining genetic information by evaluating the gene of a cancer tissue-derived cell mass in advance and of correlating the genetic information with the drug candidate compound.

The present invention also relates to a drug obtained by any one of the screening methods described above.

The present invention also relates to a cancer tissue-derived cell mass in a dormant state in vitro, which maintain such state for at least three days that the cell mass does not substantially grow, does not substantially cause cell death, in a state of a glucose consumption of 10% or less and an oxygen consumption of 30% or less, in comparison with a normal state, and can be present in a state able to recover the proliferation ability, glucose consumption and oxygen consumption by culturing it under the conditions of at least 1 ng/ml to 200 ng/ml of a growth factor, the cancer tissue-derived cell mass being a substantially spherical shape or spheroidal form and not containing substantially cells other than cancer cells.

The dormant state maybe a state where phosphorylation of Akt and/or S6 is reduced, and/or the amount of c-Myc protein is decreased to 10% or less compared to the normal state.

By utilizing the dormant state of the cancer tissue-derived cell mass of the present invention, it is possible to evaluate various treatments with drugs and the like. Since similar behavior which is likely to occur in the living body can be known in advance in vitro by this evaluation method with drugs, the effect can be predicted before administration of the drug to the patient in vivo and before application of various treatments. Therefore, with the use of cancer tissue-derived cell mass which is prepared from a cancer tissue from an individual patient, it is possible to quickly and accurately establish an optimal therapeutic method corresponding to, not uniformly, the individual patient.

Moreover, in the present invention, a method for screening anticancer agents can be performed by applying a cancer tissue-derived cell mass or an aggregated cancer cell mass to a candidate drug such as anticancer agents and the like and examining the sensitivity. It is possible to effectively screen anticancer agents effective against individual patients or a specific population having some common characteristics.

### BRIEF DESCRIPTION OF THE DRAWINGS

FIG. 1 is a drawing showing formation of a cancer tissue-derived cell mass of the present invention;

FIG. 2 is a drawing showing a cancer tissue-derived cell mass of the present invention;

FIGS. 3A-3C show a proliferation state when a cancer tissue-derived cell mass of the present invention is cultured; FIG. 3A shows the effect of oxygen and growth factors on proliferation of a cancer tissue-derived cell mass, which is derived from human colon cancer (sample C45) of the present invention; FIG. 3B shows the effect of oxygen and growth factors on proliferation of a cancer tissue-derived cell mass, which is derived from human colon cancer (sample CB3) of the present invention; FIG. 3C shows the effect of oxygen and growth factors on proliferation of a cancer tissue-derived cell mass, which is derived from human colon cancer (sample C111) of the present invention;

FIGS. 4A-4B show a proliferation state when a cancer tissue-derived cell mass of the present invention is cultured; FIG. 4A is a graph showing the effect of growth factors on proliferation of a cancer tissue-derived cell mass (sample C45) of the present invention under 1% oxygen; FIG. 4B is a picture showing the effect of growth factors on proliferation of a cancer tissue-derived cell mass (sample C45) of the present invention under 1% oxygen;

FIGS. 5A-5B show oxygen consumption and glucose consumption when a cancer tissue-derived cell mass of the present invention is cultured;

FIG. 5A is a graph showing oxygen consumption of a cancer tissue-derived cell mass (C45) of the present invention; FIG. 5B is a graph showing glucose consumption of a cancer tissue-derived cell mass (C45) of the present invention;

FIGS. 6A-6C show the result of an in vitro drug sensitivity test using a cancer tissue-derived cell mass of the present invention; FIG. 6A shows the result of an in vitro drug (SN38) sensitivity test using a cancer tissue-derived cell mass of the present invention; FIG. 6B shows the result of an in vitro drug (5FU) sensitivity test using a cancer tissue-derived cell mass of the present invention; FIG. 6C shows the result of an in vitro drug (oxaliplatin) sensitivity test using a cancer tissue-derived cell mass of the present invention;

FIGS. 7A-7B show the result of examining the relationship between a cancer tissue-derived cell mass of the present invention and an oxygen partial pressure; FIG. 7A shows the effect of oxygen on a cancer tissue-derived cell mass of the present invention in the absence of growth factor; FIG. 7B shows the effect of oxygen on a cancer tissue-derived cell mass of the present invention and an oxygen partial pressure in the presence of growth factor;

FIG. 8 is a drawing showing the result of a radio sensitivity test using a cancer tissue-derived cell mass of the present invention;

FIGS. 9A-9C show the result of examining the relationship of a cancer tissue-derived cell mass of the present invention with an oxygen partial pressure and a growth factor; FIG. 9A shows the effect of oxygen and growth factor on a cancer tissue-derived cell mass derived from human lung cancer (sample LB18) of the present invention; FIG. 9B shows the effect of oxygen and growth factor on a cancer tissue-derived cell mass derived from human lung cancer (sample LB30) of the present invention;

FIG. 9C shows the effect of oxygen and growth factor on a cancer tissue-derived cell mass derived from human lung cancer (sample LC62) of the present invention;

FIG. 10 is a drawing showing the result of examining the relationship of a cancer tissue-derived cell mass of the present invention with an oxygen partial pressure and a growth factor; and

FIG. 11A shows the result of an in vitro drug (SN38) sensitivity test using a cancer tissue-derived cell mass of the present invention;

FIG. 11B shows the result of an in vitro drug (5-FU) sensitivity test using a cancer tissue-derived cell mass of the present invention;

FIG. 11C shows the result of an in vitro drug (oxaliplatin) sensitivity test using a cancer tissue-derived cell mass of the present invention.

### DESCRIPTION OF PREFERRED EMBODIMENTS

The cancer tissue-derived cell mass according to the present invention may be those obtained by culturing an isolated product that is isolated from a cancer tissue obtained from an individual as a mass containing three or more cancer cells and that can retain a proliferation ability in vitro. That is, as used herein, the “mass containing three or more cancer cells” is to be preferably separated while holding at least cell adhesion between cancer cells. But not limited to, a state in which an intercellular adhesion molecule such as cadherin and the like is interposed is particularly preferred.

Here, the expression of “an isolated product that is isolated from a cancer tissue obtained from an individual as a mass containing three or more cancer cells” means an isolated product obtained by treatment of a cancer tissue of a cancer that has occurred in a living body and containing three or more cancer cells, preferably eight or more cancer cells, or 20 or more cancer cells. Such an isolated product does not include a product isolated to single cells as well as does not include a composition that has been once separated to single cells and has been then reconstructed. However, this isolated product includes not only a product obtained just after isolation from a living body, but also a product that is kept in, for example, a physiological saline solution for a certain period of time, or a product after freezing or cryopreservation.

The “cancer tissue obtained” from an individual refers to a cancer tissue obtained by surgical removal, etc., as well as a cancer tissue obtained with a needle or an endoscope so that it is possible to handle it in vitro for a tissue examination.

The expression of “a culture of an isolated product that is isolated from a cancer tissue obtained by isolation from an individual as a mass containing three or more cancer cells” refers to a product obtained by culturing in vitro an isolated product obtained by isolation from a cancer tissue of a cancer that has occurred in a living body as a mass containing three or more cancer cells. The culturing time is not particularly limited, and the culture may include a culture that is allowed to be present in a medium even for a short time. This culture often takes an almost spherical or spheroidal form after being cultured for a certain period of time, preferably for three hours or more. The culture as described herein includes a culture with an almost spherical or spheroidal form after such a certain period of time. In addition, the culture as described herein includes a culture with an irregular form obtained by dividing such an almost spherical or spheroidal form, and a culture having an almost spherical or spheroidal form after further culture. These cultures are all included in the cancer tissue-derived cell mass in accordance with the present invention.

The various treatment means in the present invention refer to means employed for various treatments such as drug administration, radiation exposure, photoirradiation used for photochemotherapy, treatment in immunotherapy, thermotherapy, and the like. The term “drug” in the present invention refers to any physiologically active existing or unknown substances that can stimulate a living body or cells in addition to those used for the treatment of cancers. In addition to low molecular weight compounds, the drug as used herein includes peptide formulations, nucleic acid medicines, and proteins such as antibodies.

The expression of “can retain a proliferation ability” means that the cancer tissue-derived cell mass according to the present invention can retain a proliferation ability in vitro for 10 or more days, preferably 13 or more days, and more preferably 30 or more days, in a cell culture medium under cell culture conditions of a temperature of 37° C. and a 5% CO₂-incubator.

Although such a cancer tissue-derived cell mass can retain a proliferation ability while continuing to culture without mechanical division for a period of 10 or more days, preferably 13 or more days, and more preferably 30 or more days, the proliferation ability can be retained substantially indefinitely by mechanically dividing the cell mass periodically during the culture.

The mechanical division of the cell mass can be performed using a surgical scalpel, knife, scissors, as well as an ophthalmic pointed knife. Alternatively, the mechanical division can also be performed by attaching an injection needle to a syringe and repeating suction and discharge of the cancer tissue-derived cell mass together with a culture fluid. For example, a 1 ml syringe and a 27 G needle are, but not limited to, preferably used in the present invention.

Here, the medium for culture of the cancer tissue-derived cell mass according to the present invention is not particularly limited, but an animal cell culture medium (i.e. a culture medium for mammal cells) is preferably used. Especially preferably, a serum-free medium for stem cell culture is used. Such a serum-free medium is not limited at all so long as it can be used for stem cell culture. The serum-free medium refers to a medium which does not contain a non-adjustable and non-purified serum, and it can be used after addition of a purified blood-derived component or an animal tissue-derived component (e.g., a growth factor).

The serum-free medium of the present invention can be prepared using a medium used for mammal cell culture as a basal medium. The basal medium includes, for example, BME medium, BGJb medium, CMRL 1066 medium, Glasgow MEM medium, Improved MEM Zinc Option medium, IMDM medium, Medium 199 medium, Eagle MEM medium, aMEM medium, DMEM medium, RPMI 1640 medium, Fischer's medium, and a combination thereof.

It is possible to culture the cancer tissue-derived cell mass of the present invention by adding a serum substitute to such a serum-free medium. The serum substitute maybe those appropriately containing, for example, albumin, amino acids (e.g., non-essential amino acids), transferrin, fatty acids, insulin, collagen precursor, trace elements, 2-mercaptoethanol or 3′-thiolglycerol, or an equivalent thereof.

In the culture method of the present invention, a commercially available serum substitute can also be used. Examples of such a commercially available serum substitute include a knockout serum replacement (KSR, manufactured by Life Technologies Japan Ltd.), a Chemically-defined Lipid concentrated (manufactured by Gibco Company), and a Glutamax (manufactured by Gibco Company).

The medium used for culturing the cancer tissue-derived cell mass according to the present invention may also contain vitamins, growth factors, cytokines, antioxidants, pyruvic acid, buffers, inorganic salts, etc.

In particular, any serum-free media, such as a serum-free medium containing EGF and bFGF, for example, a serum-free medium containing a serum substitute [e.g. knockout serum replacement (KSR, manufactured by Invitrogen Corporation)] and bFGF can be preferably used. The content of the serum substitute is preferably 10 to 30% w/v based on the whole medium.

Such a medium is not limited, but a commercially available product includes a STEMPRO serum-free medium (Gibco Company) for human ES cells.

A culture vessel used for culturing the cancer tissue-derived cell mass can include, but not particularly limited to, for example, flask, flask for tissue culture, dish, petri dish, dish for tissue culture, multi dish, micro plate, micro-well plate, multi plate, multi-well plate, chamber slide, schale, tube, tray, culture bag, and roller bottle, as long as the vessel is generally capable of culturing an animal cell therein.

The culture vessel can be cellular non-adhesive, and a three-dimensional culture is preferably performed in a medium in which a cell supporting substrate (e.g. an extracellular matrix (ECM), etc.) should be co-present. The cell supporting substrate can be any material intended to attach the cancer tissue-derived cell mass. Examples of such a cell supporting substrate include Matrigel using an extracellular matrix, such as collagen gel, gelatin, poly-L-lysine, poly-D-lysine, laminin, fibronectin, etc. These conditions are preferably used particularly for the proliferation of the cancer tissue-derived cell mass according to the present invention.

Other culture conditions can be appropriately set. For example, the culture temperature can be, but not limited to, about 30 to 40° C., and most preferably 37° C. The CO₂ concentration can be, for example, about 1 to 10% and preferably about 2 to 5%.

The cancer tissue-derived cell mass according to the present invention can be cultured in such a medium under such a culture condition. Furthermore, for the culture of the cancer tissue-derived cell mass, coculture with other cells may be desirable in some cases depending on individual properties, or a special additional supplement such as hormones may be necessary in some cases.

Specifically, coculture may be performed in the presence of feeder cells. For the feeder cells, stromal cell and the like such as fetal fibroblast may be used. Specifically, NIH3T3 and the like are preferable, but not limited to them.

Alternatively, in the case of a specific kind of breast cancer, uterine cancer, and prostate cancer, culture of such a cancer cell mass is performed preferably in the presence of a hormone. Specifically the hormone includes, but not limited to, estrogen for breast cancer, progesterone for uterine cancer, and testosterone for prostate cancer, and culture conditions can be conveniently adjusted while adding various hormones. In addition, hormone dependence of a cancer derived from a patent is understood by examining how behavior after culture of the cancer tissue-derived cell mass is changed in the presence of such a hormone. As a result, effectiveness of an anti-hormone therapy may be predicted.

It is also possible to culture the cancer tissue-derived cell mass according to the present invention by floating culture. In the floating culture, the cancer tissue-derived cell mass is cultured in a medium under a non-adhesive-condition to a culture vessel. Such a floating culture includes an embryoid culture method (see Keller et al., Curr. Opin. Cell Biol. 7, 862-869 (1995)), and an SFEB method (for example, Watanabe et al., Nature Neuroscience 8, 288-296 (2005); International Publication No. WO 2005/123902). The floating culture maybe used in the production and maintenance of a stable tissue culture-derived cell mass, which cell mass has, but not particularly limited to, an almost spherical shape and has a basement membrane in some cases.

The cancer tissue-derived cell mass according to the present invention includes an isolated product just after isolation from the cancer tissue-derived cell mass of an individual or its cultured product, and further a product after freezing or cryopreservation. After such separation, the culture may be carried out preferably for three hours or more, more preferably for 10 hours or more, and still more preferably for 24 hours or more.

The cancer cells constituting a cancer tissue-derived cell mass and contained in a cancer tissue-derived cell mass are composed of 3 or more cancer cells, preferably 8 or more cancer cells, more preferably 10 or more cancer cells, still more preferably 20 or more cancer cells, and most preferably 50 or more cancer cells. In the case where the cancer tissue-derived cell mass according to the present invention is an isolated product, it includes preferably 1000 cancer cells or less, and more preferably about 500 cancer cells or less. In the case of a culture after culturing the isolated product, it is possible to increase the number of the cancer cells by culture. However, even the culture contains preferably 10,000 cancer cells or less, and more preferably 5000 cancer cells or less.

In the present invention, the cancer tissue-derived cell mass may also mean any of compositions including a cancer tissue-derived cell mass or a plurality of cancer tissue-derived cell masses.

The term of “cancer cell” as used in the present invention is used in the sense commonly used, and refers to a cell where an order to be seen in normal cells is disordered, such as unrestricted division/proliferation and escape from apoptosis in a living body. More particularly, the term refers to a cell which has lost a control function for cell proliferation or refers to an extremely attenuated cell, and a cell which has typically acquired an infinite proliferation ability at a high frequency of 80% or more, many of which also have an ability of invasion and metastasis, and, as a result, are regarded as a malignant neoplasm that causes the death particularly in a mammal including humans.

In the present invention, the kind of the tissue derived from a cancer is not particularly limited, but it can be derived from cancers that occur in an animal including a mammal, such as a lymphoma, a blastoma, a sarcoma, a liposarcoma, a neuroendocrine tumor, a mesothelioma, a neurinoma, a meningioma, an adenoma, a melanoma, a leukemia, and a malignant lymphoma, etc., and particularly preferably a carcinoma that occurs in mammalian epithelial cells. Examples of such a carcinoma that occurs in mammalian epithelial cells include a non-small cell lung cancer, a hepatocyte cancer, a bile duct cancer, an esophagus cancer, a stomach cancer, a colorectal cancer, a pancreatic cancer, a cervical cancer, an ovarian cancer, an endometrial cancer, a bladder cancer, a pharyngeal cancer, a breast cancer, a salivary gland cancer, a kidney cancer, a prostate cancer, a labia cancer, an anal cancer, a penis cancer, a testicular cancer, a thyroid cancer, and a head and neck cancer. The animal including a mammal includes, but not particularly limited to, an animal belonging to Primates such as monkey and human, an animal belonging to Rodentia such as mouse, squirrel, and rat, an animal belonging to Lagomorpha, and an animal belonging to Carnivora such as dog and cat.

Among them, the cell mass of the present invention is particularly preferably derived from, but not limited to, a colon cancer tissue, an ovarian cancer tissue, a breast cancer tissue, a lung cancer tissue, a prostate cancer tissue, a kidney cancer tissue, a bladder cancer tissue, a pharyngeal cancer tissue, or a pancreatic cancer tissue.

Isolation of the cancer tissue obtained from a cancer that occurs in a living body is not limited, but includes an enzymatic treatment of a cancer tissue obtained from an individual.

The enzymatic treatment can be a treatment using one member of enzymes selected from collagenase, trypsin, papain, hyaluronidase, C. histolyticum neutral protease, thermolysin, and dispase, or a combination of two or more enzymes thereof. Other matrix metalloproteinase may also be used. The conditions for such an enzymatic treatment maybe as follows: in an isotonic salt solution (e.g. PBS or Hanks' balanced salt solution) buffered at a physiologically acceptable pH (e.g. about pH 6 to 8, preferably about pH 7.2 to 7.6) at for example about 20 to 40° C., preferably at about 25 to 39° C., fora time sufficient to degrade a connective tissue, for example, for about 1 to 180 minutes, preferably 30 to 150 minutes, with a sufficient concentration for such degradation, for example, about 0.0001% to 5% w/v, preferably about 0.001% to 0.5% w/v.

The conditions for such an enzymatic treatment include, but not limited to, a treatment with a mixed enzyme containing collagenase. For example, the enzymatic treatment includes a treatment with a mixed enzyme including one or more proteases selected from the group consisting of C. histolyticum neutral protease, thermolysin, and dispase, and one or more collagenases selected from the group consisting of collagenase I, collagenase II, and collagenase IV. Combination of collagenase and dispase is especially preferred. Alternatively, only either of collagenase and dispase may be preferred. It is preferable to digest only interstitial collagen and fibronectin by an enzymatic treatment and not to digest a protein that is involved in epithelial cell-cell adhesion. In particular, it is preferable not to completely digest a cell adhesion molecule such as cadherin.

Such a mixed enzyme is not limited, but includes, for example, Liberase Enzyme Blend (manufactured by Roche Applied Science) and particularly, Liberase DH Research Grade is preferably used.

The cancer tissue-derived cell mass according to the present invention includes a population of three or more cancer cells and may take an almost spherical or spheroidal form.

The cell mass may contain, but not limited to, a basement membrane-like material present in the circumference of the cancer cell population.

Cancer cells forming a population that constitutes one cancer tissue-derived cell mass can be preferably a population including substantially pure cancer cells only and can be more preferably a population including pure cancer cells only. That is, in the population constituting the cancer tissue-derived cell mass, 80% or more, preferably 90% or more, more preferably 95% or more, and most preferably 99% or more of the cell mass are composed of cancer cells. Here, the cancer cells forming the population of the cancer tissue-derived cell mass often have one or more surface antigens selected from the group consisting of, but not particularly limited to, CD133, CD44, CD166, CD117, CD24 and ESA on the cell surface. The CD133, CD44, CD166, CD117, CD24, and ESA are surface antigens that are generally expressed in the cells such as leucocytes (e.g. lymphocytes), fibroblasts, epithelial cells, and tumor cells. These surface antigens are involved in various signal transduction in addition to a function of cell-cell adhesion and cell-matrix adhesion, and can also be surface markers for various stem cells. In addition, for example, in the case of a cancer tissue-derived cell mass that is derived from a colon cancer tissue, the cancer cell to be included therein is not limited particularly, but may also express CD133.

When cell groups “express” surface antigens such as CD133 in the present invention, the term “express” means a state where 80% or more of the cells present in the cell groups, preferably 90% or more of the cells present in the cell groups, more preferably 95% or more of the cells present in the cell groups, most preferably 99% or more of the cells present in the cell groups, or a substantially whole of the cells present in the cell groups represent surface antigens.

In the present specification, the term “basement membrane-like material” refers to, but not limited to, a substance that contains preferably at least one member selected from proteoglycans, such as collagen, laminin, nidogen and heparan sulfate proteoglycan; and glycoproteins, such as fibronectin. In the present invention, a basement membrane-like material containing laminin is preferable.

The presence of laminin can be detected, for example, by contacting an antibody that recognizes laminin (e.g. an anti-laminin antibody produced in a rabbit host; Sigma-Aldrich Corporation) with a cancer tissue-derived cell mass, and measuring the antigen-antibody reaction.

Moreover, it is also possible to use a specific antibody that can specify even the kind of the laminin. For example, the presence of laminin-5 can be detected, for example, by contacting an antibody that is reactive particularly to the above inherent γ2-chain or its fragment, with a cancer tissue-derived cell mass, and measuring the reaction with the antibody.

In the cancer tissue-derived cell mass according to the present invention, it is desirable that a thin filmy basement membrane-like material is formed in a size of about several nanometers, or about 40 to 120 nm, according to the size of masses, but the size is not limited to them.

The size of the cancer tissue-derived cell mass according to the present invention also includes, but not limited to, an irregular form with a particle size or a Mean Volume Diameter of particle of about 8 μm to 10 μm, as well as further includes a particle size of 1 mm or more of the cell mass that has been grown up greatly after incubation. The diameter of the cell mass is preferably 40 μm to 1000 μm, more preferably 40 μm to 250 μm and further more preferably 80 μm to 200 μm.

The cancer tissue-derived cell mass according to the present invention often has one or more arrangements particularly selected from the group consisting of, but not particularly limited to, palisade arrangement, sheet arrangement, multilayer arrangement, and syncytial arrangement.

The cancer tissue-derived cell mass according to the present invention may be prepared typically by a process which includes the steps of treating a fragmented product of a cancer tissue removed from a living body, with an enzyme; and selecting and collecting a mass containing three or more cancer cells from enzymatic treatment products.

Moreover, the cancer tissue-derived cell mass according to the present invention may be prepared by, but not limited to, a process including the step of culturing the thus collected component for three hours or more.

At first the cancer tissue removed from a living body can be fragmented as it is, or the cancer tissue is first maintained in a medium for animal cell culture before fragmentation. The medium for animal cell culture includes, but not particularly limited to, Dulbecco's MEM (DMEM F12, etc.), Eagle's MEM, RPMI, Ham's F12, alpha MEM, and Iscove's modified Dulbecco's medium. In this case, floating culture is preferably carried out in a culture vessel which is not adhesive to cells.

It is also preferable to wash the cancer tissue in advance for fragmentation. Such a washing can be carried out using, but not limited to, a buffer solution such as acetic acid buffer solution (acetic acid+sodium acetate), phosphoric acid buffer solution (phosphoric acid+sodium phosphate), citric acid buffer solution (citric acid+sodium citrate), boric acid buffer solution, tartaric acid buffer solution, Tris buffer solution, and phosphate-buffered saline. In the present invention, washing of the tissue can be performed particularly preferably with HBSS. As for the number of times of the washing, once to three times are suitable.

The fragmentation can be performed by dividing the tissue after washing, with use of a knife, scissors, or a cutter (manual operation and automatic operation). The size and form after fragmentation are not particularly limited, but the fragmentation may be performed at random. The tissue is fragmented to a uniform size, preferably 1 mm to 5 mm square, more preferably 1 mm to 2 mm square.

The fragmented product thus obtained is then subjected to an enzymatic treatment. Such an enzymatic treatment can be a treatment using one member of enzymes selected from collagenase, trypsin, papain, hyaluronidase, C. histolyticum neutral protease, thermolysin, and dispase, or a combination of two or more enzymes thereof. The conditions for such an enzymatic treatment may be as follows: in an isotonic salt solution (e.g. PBS or Hank's balanced salt solution) buffered at a physiologically acceptable pH (e.g. about pH 6 to 8, preferably about pH 7.2 to 7.6) at for example about 20 to 40° C., preferably at about 25 to 39° C., for a time sufficient to degrade a connective tissue, for example, about 1 to 180 minutes, preferably about 30 to 150 minutes, with a sufficient concentration for such degradation, for example, about 0.0001% to 5% w/v, preferably about 0.001% to 0.5% w/v.

The conditions for this enzymatic treatment include, but not limited to, a treatment using a mixed enzyme containing, for example, collagenase. More preferably, the enzymatic treatment includes a treatment with a mixed enzyme containing one or more protease selected from the group consisting of C. histolyticum neutral protease, thermolysin, and dispase, and one or more collagenase selected from the group consisting of collagenase I, collagenase II, and collagenase IV. Combination of collagenase and dispase is particularly preferred. It is preferable to digest only interstitial collagen and fibronectin by an enzymatic treatment and not to digest a protein that is involved in epithelial cell-cell adhesion, such as cadherin.

Such a mixed enzyme is not limited, but includes Liberase Enzyme Blend (manufactured by Roche Applied Science) and the like.

Among the enzymatic treatment products obtained in this way, it is preferable to select and collect a mass containing three or more cancer cells. The process for such selection and collection is not particularly limited, but any process well-known to those skilled in the art for assorting the size can be used.

Of the methods for assorting the size, a simple and easy process is a visual observation, a classification with a phase contrast microscope, or a classification with a sieve, but the classification method is not particularly limited so long as it is a classification with a particle size available for a person skilled in the art. When a sieve is used, it is preferable to collect a component which passes through a sieve with a mesh size (a diameter of one mesh) of 500 μm and does not pass through a sieve with a mesh size of 20 μm. It is more preferable to collect a component which passes through a sieve with a mesh size (a diameter of one mesh) of 250 μm and does not pass through a sieve with a mesh size of 40 μm.

Here, the mass containing three or more cancer cells, which is a subject for selection, is a cancer tissue-derived cell mass according to the present invention and has a certain range of sizes. The term of “a certain range of sizes” includes small ones with a Mean Volume Diameter of particle of about 8 μm to 10 μm. When the cell mass is in an almost sphere form, it has a diameter of 20 to 500 μm, preferably 30 to 400 μm, and more preferably 40 to 250 μm. When the cell mass is in an spheroidal form, it has a long diameter of 20 to 500 μm, preferably 30 to 400 μm, and more preferably 40 to 250 μm. When the cell mass is in an irregular form, it has a Mean Volume Diameter of particle of 20 to 500 μm, preferably 30 to 400 μm, and more preferably 40 to 250 μm. The measurement of the Mean Volume Diameter of particle can be performed by evaluating a particle size distribution and a particle shape using a CCD camera attached to a phase contrast microscope (IX70; manufactured by Olympus Corporation).

The culture product of the isolated product that is a component obtained in this way by selection and collection is a cancer tissue-derived cell mass according to the present invention. The cultured product may be those wherein the isolated product as a component after selection and collection has been present in a medium for a short time, or those which are in an almost sphere or spheroidal form after culture for a period of time, for example, at least three hours, preferably 10 to 36 hours, and more preferably 24 to 36 hours. The culture time may be over 36 hours, several days, 10 or more days, 13 or more days, or 30 or more days.

The culture may be performed in a medium for a long time without any mechanical division, but a proliferation ability can also be retained for a substantially infinite time period by a mechanical division periodically on the way of culture.

In the present invention, it is possible to easily obtain a composition in which a plurality of such cancer tissue-derived cell masses are present, and a substantially pure aggregate of such a cancer tissue-derived cell mass is highly useful in various applications. The composition including an aggregate composed of a plurality of the cancer tissue-derived cell masses contains, for example, 5 or more, preferably 10 or more, and preferably 50 or more, of the cancer tissue-derived cell masses in an almost pure form. If the aggregate composed of a plurality of the cancer tissue-derived cell masses is pure, this means that other cells derived from the tissue are not contained in the composition. That is, 80% or more, preferably 90% or more, more preferably 95% or more, and most preferably 99% or more, of the cancer cells is present in the composition containing the cancer tissue-derived cell mass.

The cancer tissue-derived cell mass of the present invention, even if it includes, for example, 10 or less cancer tissue-derived cell masses (equivalent to 1000 or less cells) with a diameter of 100 μm, has a high engraftment rate in the transplantation in different species of animals. Therefore, the cancer tissue-derived cell mass of the present invention makes it possible to examine a cancer tissue more strictly, evaluate drug sensitivity, or evaluate therapeutic embodiments including a radiation therapy, phototherapy, and immunotherapy.

It is possible to cryopreserve the cancer tissue-derived cell mass of the present invention, and it is possible to retain the proliferation ability in a normal storage state.

The cancer tissue-derived cell mass of the present invention is once formed as a cancer tissue-derived cell mass and then unicellularized to reconstitute an aggregate formed by causing the mutual aggregation of three or more cells as a whole among the single cells; or causing the mutual aggregation of three or more cells as a whole among some cell populations that have not been separated completely into individual cells; or causing the aggregation of three or more cells as a whole between the individual cells and the some cells that have not been completely separated; or a cultured product thereof.

Here, the expression of “unicellularizing a cancer tissue-derived cell mass or a cancer tissue obtained from an individual” means that a separation treatment is applied until at least a part of the cancer tissue-derived cell mass or the obtained cancer tissue is allowed to be separated in vitro so that single cells are contained to some extent. Thus, typically after such a treatment, the expression of “unicellularizing” as used herein corresponds to even in a case where some cells separated into single cells are present and some cells not separated into individual cells are present in a mixed state. Even in such a case with a mixed state, it refers to “unicellularizing” as defined in the present invention. At this time, those that are mixed in a state not being separated into individual cells include a cell population with up to ten cells, and preferably a cell population with two or three cells.

The expression of “aggregation of three or more cells” refers to a state including multiple cells of at least 3 wherein individual cells obtained by unicellularizing a cancer tissue obtained from a cancer that occurs in vivo or from a cancer tissue-derived cell mass that has been found by the present inventor(s) are mutually gathered; or some cell populations that have not been separated into individual cells are mutually gathered; or combinations thereof are mutually gathered.

If the cancer tissue obtained from the cancer tissue-derived cell mass is subjected to a unicellularization treatment, it includes, but not limited to, an enzymatic treatment of the cancer tissue-derived cell mass.

The enzymatic treatment may be a treatment using typically one kind selected from trypsin, dyspase, and optionally collagenase, papain, hyaluronidase, C. histolyticum neutral protease, thermolysin, and dispase or a combination of two or more enzymes thereof. The enzymatic treatment conditions may be such that the treatment is carried out in a buffered isotonic salt solution (for example, PBS or Hank's balanced salt solution) having a physiologically acceptable pH of about 6 to 8, and preferably of about 7.2 to 7.6, at for example about 20 to 40° C., and preferably at about 25 to 39° C., for a sufficient time to degrade the connection tissue, for example, about 1 to 180 minutes, and preferably 30 to 150 minutes, at a concentration sufficient for such a purpose, for example, about 0.0001 to 5% w/v, and preferably about 0.001% to 0.5% w/v.

This enzymatic treatment may be, but is not limited to, typically a single treatment with trypsin or dyspase.

After the unicellularization treatment, the resulting cells include individually separated cells as well as cells that have not been completely separated into individual cells.

Such cells may be aggregated as they are, but they may be treated with the addition of, for example, an agent to promote the cell aggregation or an agent to suppress the cell death. Examples of such agents include enzyme inhibitors associated with the cell death, such as ROCK inhibitors and caspase inhibitors.

ROCK refers to Rho-associated coiled-coil kinase (ROCK: GenBank accession number: NM_{—}005406), is one of the main effector molecules of Rho GTPase, and is known to control various physiological phenomena (also referred to as Rho-binding kinase). Examples of the ROCK inhibitor include Y27632, and in addition, Fasudil (HA1077), H-1152, Wf-536 (all available from Wako Pure Chemical Industries, Ltd.), and derivatives thereof, and antisense nucleic acids against ROCK, and RNA interference-inducing nucleic acids, and vectors containing these nucleic acids.

The treated product that is separated into a population including single cells or 10 or less cells by an enzymatic treatment including a trypsin treatment (it is, but is not limited to, a treatment with 0.25% trypsin-EDTA at 37° C. for 5 minutes) is seeded in a 96-well culture plate at a low density (for example, 500 cells/0.32 cm², medium volume: about 0.15 ml) prior to aggregation. The ROCK inhibitor may be added in a concentration of about 1 to 100 μM, and preferably about 10 μM, to a maintenance culture solution immediately or several days after culturing.

Such an aggregated product can be cultured in vitro. The culturing time may not be particularly limited as long as the aggregated product is present in the culture medium even for a little time. Such a cultured product often exhibits a substantially spherical or spheroidal form by culturing the cultured product for a fixed period of time of preferably 3 hours or more. The cultured product herein also includes not only a cultured product having a substantially spherical or spheroidal form after the fixed period of time but also an irregular cultured product before reaching such a form. Further, the cultured product as used herein includes an irregular form obtained by further dividing the cultured product having a substantially spherical or spheroidal form and a cultured product having a substantially spherical or spheroidal form obtained by further culture.

In the reconstitution of the aggregated mass from a cancer tissue using a cancer tissue-derived cell mass, such a cancer tissue-derived cell mass as it is can be fractionated to form single cells by an enzymatic treatment, and it is preferred to perform fragmentation before such an enzymatic treatment. It is possible to maintain the cell in a medium for animal cell culture before fragmentation. The medium for animal cell culture includes, but not particularly limited to, Dulbecco's MEM (DMEM F12, etc.), Eagle's MEM, RPMI, Ham's F12, alpha MEM, and Iscove's modified Dulbecco's medium. In this case, floating culture is preferably carried out in a culture vessel which is non-adhesive to cells.

Such an enzymatic treatment of the fragmented product may be a treatment using mainly trypsin as described above. The conditions therefor may be as follows: at 20 to 45° C. for several minutes to several hours.

Then, the cells in the unicellularized product thus obtained in this way are allowed to mutually aggregate into 3 or more cells. Preferably, prior to such an aggregation, it is possible to rapidly add a ROCK inhibitor to single cells.

Here, the aggregate containing 3 or more cells, obtained by the aggregation, is the aggregated cancer cell mass of the present invention and has a certain range of sizes. The certain range of sizes includes those with a small volume average particle diameter of about 8 μm to 10 μm. When the cell mass is an almost sphere form, it has a diameter of 20 μm or more and 500 μm or less, preferably 30 μm or more and 400 μm or less, and more preferably 40 μm or more and 250 μm or less. When the cell mass is in a spheroidal form, it has a long diameter of 20 μm or more and 500 μm or less, preferably 30 μm or more and 400 μm or less, and more preferably 40 μm or more and 250 μm or less. When the cell mass is in an irregular form, it has a volume average particle diameter of 20 μm or more and 500 μm or less, preferably 30 μm or more and 400 μm or less, and more preferably 40 μm or more and 250 μm or less. The measurement of the volume average particle diameter can be carried out by evaluating the particle diameter distribution and the particle form using a phase contrast microscope attached with a CCD camera (IX 70; manufactured by Olympus Corporation).

Both the aggregated product and its cultured product, which are obtained in this way, are the cancer tissue-derived cell mass of the present invention. The cultured product may be those in which the separated product as a component after selection and collection has been present in a culture medium for a short time, or those which are in the form of a substantially sphere form or a substantially spheroidal form after culture for a period of time, for example, at least 3 hours, preferably 10 hours or more and up to 36 hours, and more preferably 24 to 36 hours. The culture time may be over 36 hours, several days, 10 days or more, 13 days or more, or 30 days or more.

The culturing may be carried out in a culture medium for a long time without any mechanical division, but the proliferation ability can also be retained for a substantially infinite time period by mechanical division periodically on the way of culture.

The cancer tissue-derived cell mass of the present invention thus obtained shows an in vitro behavior similar to a cancer tissue in a living body and can be stably cultured while retaining its proliferation ability. In particular, an aggregated form in which a plurality of such cancer tissue-derived cell masses are present can be easily prepared and thus is very useful in the present invention.

Next, such a cancer tissue-derived cell mass can be induced in vitro to a dormant state that is also considered to exist in vivo.

Preferably, the cancer tissue-derived cell mass is usually cultured in an in vitro environment of an oxygen concentration of 16% or more under the conditions of a growth factor of about 1 ng/ml to 200 ng/ml to the whole medium. The dormant state of the cancer tissue-derived cell mass of the present invention may be induced by placing a cancer tissue-derived cell mass in a more hypoxic state as well as in a lower level of growth factors than under normal conditions. Alternatively, the dormant state can also be induced by only hypoxia in some cases. More specifically, culturing the cancer tissue-derived cell mass in a hypoxic state refers to culture wherein the oxygen concentration in the culture environment is 0 to 15% by volume of the total environment, more preferably 0.1 to 5% by volume, and most preferably 0.1 to 2% by volume. Culturing the cancer tissue-derived cell mass under the condition of low levels of growth factors refers to culture wherein the concentration of the growth factors is 0.9 ng/ml or less, preferably 0.5 ng/ml or less of the total medium. Alternatively, even when 1 ng/ml or more of the growth factor is present in the medium and if a growth factor inhibitor corresponding to the growth factors contained is included in the medium, a state where the growth factor does not function as a result or a state where the function of the growth factor is reduced as a result can be included in the state of low levels of growth factors in the present invention.

Here, growth factors are called a cell proliferation factor or a growth factor, and refers to a substance that acts on the regulation of various cytological/physiological processes and functions as signal substances between cells by specifically binding to receptor proteins on the surface of target cells.

Typical examples of the growth factor includes, but not limited to, epidermal growth factor (EGF), insulin-like growth factor (IGF), transforming growth factor (TGF), nerve growth factor (NGF), brain-derived neurotrophic factor (BDNF), vascular endothelial growth factor (VEGF), granulocyte colony stimulating factor (G-CSF), granulocyte macrophage colony-stimulating factor (GM-CSF), platelet-derived growth factor (PDGF), erythropoietin (EPO), thrombopoietin (TPO), basic fibroblast growth factor (bFGF or FGF2), hepatocyte growth factor (HGF), heregulin, Activin A, and the like. It is preferable to use HRG (NRG1) and IGF as a growth factor. FGF and Activin A are preferable as a factor required for cell maintenance. Activin A may act to suppress the growth of cells.

A hypoxic state according to the present invention can be created by adjustment of the culture environment using a carbon dioxide incubator or a cylinder for carbon dioxide, nitrogen gas or a mixed gas thereof. In addition, it is possible to create a state close to hypoxia by placing cobalt chloride, lithium chloride, deferoxamine, and the like in the medium.

Furthermore, by using a hypoxic culture kit such as a BIONIX kit (manufactured by SUGIYAMA-GEN Co., Ltd.), culturing may be carried out in a sealed bag included in such a kit while adjusting the oxygen concentration.

The growth factor inhibitor refers to an antagonist of various receptors and substances that inhibit the phosphorylation of epidermal growth factor receptor (EGFR) tyrosine kinase. Specific examples include, but not limited to, inhibitors of vascular endothelial growth factor (VEGF) receptors 1, 2, and 3, such as tivozanib, and tyrosine kinase inhibitors, such as gefitinib, erlotinib, cetuximab, panitumumab, and trastuzumab. Further, the growth factor inhibitor includes, but not limited to, PI3K inhibitors, mTOR inhibitors, MAP kinase inhibitors, and the like.

The dormant state of the cancer tissue-derived cell mass induced in this way refers to a state where there is neither substantial proliferation nor cell death in vitro and where the oxygen consumption and glucose consumption are reduced. Such dormant state can be maintained for at least 3 days or more, preferably at least 7 days or more. In the present invention, even when the dormant state of the cancer tissue-derived cell mass has been long lasting in this way, it is possible to recover the capacities for proliferation, glucose consumption, and oxygen consumption by culturing afterwards in the presence of growth factors or culturing in the presence of normal oxygen and growth factors. In addition, such a state also can refer to “proliferation ability is inherently retained”.

Here, the “substantially does not proliferate” means that the cancer tissue-derived cell mass present as a cell mass derived from cancer tissue or as a composition shows not only a state in which the growth of the cell mass has been completely suppressed, but also a state in which such growth expressed in terms of the rate of volume change is reduced to an increase of 10% or less, respectively.

The term “no substantial cell death” refers to a state where at least 70% of the cells to the total cells is survived, in addition to a state where cell death is not confirmed completely in a target population. Specifically, verification also can be performed by measuring the survival rate.

The glucose consumption can be measured by detecting the consumption of glucose contained as the medium components over time. The state where glucose consumption is reduced can be detected by measuring the concentration of glucose in the culture broth during a period of culturing. That is, when the amount of glucose in the medium is larger even if it is a little bit larger, in comparison with the amount of glucose in the similar control culturing under normal culture conditions, it can be said that such a case refers to a state where the glucose consumption is reduced. For example, when monitoring the decrease in the amount of glucose in the medium on day 10 of culture and defining the decrease rate of the amount of glucose in the culture under normal conditions as 100%, a case where the glucose consumption is 10% or less, preferably 8% or less, and more preferably 5% or less is included in the reduced state.

The oxygen consumption can be measured by a cellular respiration activity measurement apparatus. A state in which the oxygen consumption is decreased means that the oxygen consumption is suppressed approximately by 30% or less, preferably 20% or less, and more preferably about 10% or less during culture process, compared to the normal oxygen consumption.

The dormant state of the cancer tissue-derived cell mass of the present invention may be verified, but not limited to, by the reduction in phosphorylation of Akt and/or S6 and/or the decrease in the amount of c-Myc protein to 10% or less compared to the normal state.

Using such a dormant state of the cancer tissue-derived cell mass, it is able to evaluate existing or unknown drugs as anticancer agents, candidate compounds, or protein medicines such as antibodies, and nucleic acid medicines (as used herein, these terms may be collectively used as the same meaning of drugs).

Further, therapeutic applications will become possible by development of a novel method for inducing and releasing a dormant state, by development and verification of an effective application to a dormant state using various treatments such as a variety of radiotherapies, thermotherapies, photochemotherapies, and immunotherapies, and by evaluation of the efficiency in the introduction of nucleic acids into the dormant state.

As the evaluation of treatment with drugs, etc., it is possible to specifically carry out the culture under the conditions of hypoxia and low levels of growth factors and apply the drug in the step of inducing a dormant state before or at the same time of induction. Alternatively, by applying a drug or the like to a cancer tissue-derived cell mass in a dormant state obtained by culture under the conditions of hypoxia and low levels of growth factors, effects of these various treatment means can be evaluated. Furthermore, in order to evaluate various treatment means such as a drug treatment that prevents the release of dormant state, it is also possible to evaluate the effects of the various treatment means with a drug, etc. when returned to normal culture conditions from the dormant state.

The evaluation can be performed by the detection of proliferation state, by the detection of cell death, and by the detection of signal changes.

The evaluation may be possible by proliferation ability and cellular changes in a dormant state. Alternatively, the effect of the drug can also be evaluated by determining life and death of cells after the dormant state.

After a cancer tissue-derived cell mass has been cultured in a medium containing 1 ng/ml or more of a growth factor under normal conditions, effects of the target drug can also be evaluated by determining life and death of the cancer tissue-derived cell mass or detecting the proliferation state of the cancer tissue-derived cell mass.

Furthermore, after the dormant state has been maintained for at least three days, the effect of drugs can be evaluated by analyzing intracellular signal transduction.

It is also possible to obtain genetic information of the cancer tissue-derived cell mass after evaluating its gene in advance, and correlate the genetic information with the evaluation results of treatment means such as drug treatment, etc.

In the present invention, it is able to screen non-existing drugs, particularly drugs having an action mechanism different from existing one by applying a drug candidate compound on the cancer tissue-derived cell masse in a dormant state that is a state in which there is neither substantial proliferation nor substantial cell death in vitro and in which glucose consumption and oxygen consumption are reduced.

A drug screening can also be performed by culturing a cancer tissue-derived cell mass from a patient in vitro in the presence of a drug candidate compound under the conditions of hypoxia and low levels of growth factors.

The drug screening method further comprises a step of evaluation by analyzing the proliferation state of the cancer tissue-derived cell mass, determining life and death of the cancer tissue-derived cell mass, or analyzing intracellular signal transduction of the cancer tissue-derived cell mass, while adding a growth factor in 1 ng/ml or more or culturing it under the conditions of an oxygen concentration of 16% or more and a growth factor of 1 ng/ml or more.

It is also possible to obtain genetic information of the cancer tissue-derived cell mass after evaluating its gene in advance and correlate the genetic information with the drug candidate compound.

Analysis of intracellular signal transduction includes, but not limited to, an analysis of the presence and level of phosphorylation of Akt and/or ERK1/2 in cells. Akt is a serine/threonine kinase and is activated by self-phosphorylation. On the other hand, extracellular signal-regulated kinase (ERK) belongs to the family of mitogen-activated protein kinase. AKT and ERK are involved in the growth and survival of cancer cells.

As a concrete method to analyze the presence and level of phosphorylation of Akt and/or ERK1/2, there is exemplified a method for stimulating a receptor that is known to be located upstream of these enzymes in the pathway of cellular signaling and then detecting the presence and level of phosphorylation of Akt and/or ERK1/2. For example, a cancer tissue-derived cell mass or aggregated cancer cell mass is cultured in the presence of epidermal growth factor (EGF), an EGFR antibody, or an EGFR inhibitor, followed by a cell lysis processing, and the lysate can be subjected to, for example, the Western blot method. Alternatively, the cancer tissue-derived cell mass or aggregated cancer cell mass is cultured in the presence of an antibody against HER2 having a structure similar to that of EGFR and then can be subjected to the Western blot analysis in the same manner.

As used herein, genetic information may be a result of detecting the gene expression level or may be a result of examining the state of modification changes such as methylation. Measurement of a gene expression level may be performed by detecting an expression of mRNA which is a genetic transcription product, or an expression level, as well as by detecting a protein which is a translation product of the gene, or the presence of a peptide fragment thereof or its existing amount. The transcription product of the gene can be detected or measured according to the known method to specifically detect an expression of a specific gene, such as Northern blotting method, RT-PCR method, in situ hybridization method, and DNA microarray method. Alternatively, the evaluation method for medicinal agents and radioactive rays may include a process to evaluate a gene, and the process may detect the expression level of such gene. Measurement of gene expression level may be performed by detecting an expression of mRNA which is a transcription product of the gene, or an expression level, as well as by detecting the presence of a protein which is a translation product of the gene, or a peptide fragment thereof or its existing amount thereof.

### EXAMPLES

Hereinafter, the present invention will be specifically described by way of examples, but it is not limited to these examples. In addition, parts and percentages in each example are all based on a weight basis. The culture conditions below are, unless otherwise indicated, under incubator conditions of 37° C. and 5% CO₂. The centrifugal conditions are, unless otherwise specifically stated, 4° C., 1000 rpm, and 5 minutes.

### Example 1

### Preparation of Cancer Tissue-Derived Cell Mass from Human Colon Cancer-Transplanted Mice

Human colon cancer-transplanted mice were produced by a xenograft procedure as shown below.

At first a surgical resected specimen of a human tumor (colon cancer) is cut into small pieces (each about 2 mm cube) under aseptic conditions. Then, a small incision of about 5 mm was made at the back of mice (nude mice, preferably NOD/SCID mice) with a severe immunodeficiency, and a subcutaneous tissue is peeled from the animal. A tumor graft which has been prepared is subcutaneously inserted, and wound closure is performed with a skin suture clip. Some of the xenografts are observed as a subcutaneous tumor about 14 days later to three months later.

The produced mice bearing a colon cancer were bred under SPF (specific pathogen free) conditions, and when the tumor reached 1 cm in size, it was removed and collected into a 50 ml-centrifugal tube (IWAKI; 2345-050) containing 20 ml of DMEM (Gibco; 11965-092)+1% Pen Strep (Gibco; 15140-022) (both as a final concentration of 100 units/ml penicillin, 100 μg/mL).

Next, after addition of 20 ml of HBSS (Gibco; 14025-092), the tumor was washed by inverting the tube for mixing. Then, 20 ml of a fresh HBSS was added, and these procedures were repeated twice, after which time the tumor tissue was transferred to a 10 cm-cell culture dish (Cell Culture Dish) (IWAKI; 3020-100). The necrotic tissue was removed with a surgical knife on this culture dish.

The tumor xenograft from which the necrotic tissue had been removed was transferred to a fresh 10 cm-dish in which 30 ml of HBSS had been added. Then, the tumor graft was fragmented into small pieces (each about 2 mm cube) using a surgical knife.

The fragmented tumor xenograft was transferred to a 50-ml fresh centrifugal tube, centrifuged, the supernatant was discarded, and the residue was washed by inverting the tube for mixing with a 20 ml-HBSS.

The centrifuge and washing were repeated. After that, 20 ml DMEM+1% Pen Strep+0.28 U/ml (final concentration) Blendzyme 1 (Roche; 11988417001) were added and mixed. This mixture was transferred to a 100 ml-Erlenmeyer flask and treated with Liberase Blendzyme 1 (manufactured by Roche Diagnostics K.K.) in a thermostat bath of 37° C. while rotating it with a stirrer at a low speed for 2 hours.

Then, the enzymatic treatment product was collected into a 50 ml-centrifugal tube, centrifuged, and the supernatant was discarded, after which time 20 ml of HBSS was added and mixed. The mixture was passed through a stainless mesh (500 μm), and the components that passed through the filter were collected into a 50 ml-centrifugal tube, and further centrifuged. After discarding the supernatant, 1 mg/ml DNase I solution (Roche; 1284932) (10 mg/ml stock 100 μl+PBS 900 μl) was added to the residue for mixing, and the mixture was allowed to stand at 4° C. for 5 minutes. After that, 20 ml-HBSS was further added, mixed, centrifuged, and the supernatant was discarded. The residue was mixed with 20 ml HBSS, sieved stepwise in the order of 500-250-100 μm, and then passed through a cell strainer of 40 μm (BD; 352340). The cell strainer was soaked in a 10 cm-tissue culture dish (Tissue Culture Dish) containing 30 ml of HBSS, and shaken slightly to remove single cells, small cell masses of 40 μm or less, and debris. The cell strainer was transferred to another 10 cm-tissue culture dish (Tissue Culture Dish) containing 30 ml of HBSS, and the cell mass that had been trapped in the cell strainer was collected by pipetting.

In addition, the same centrifugal separation as above was repeated several times, and 4 ml StemPro hESC SFM (Gibco; A10007-01)+8 ng/ml bFGF (Invitrogen; 13256-029)+0.1 mM 2-mercaptoethanol (Wako; 137-06862)+1% PenStrep+25 μg/ml amphotericin B (Wako; 541-01961) were added to the resulting components, and mixed. The mixture was transferred to a 6 cm-non-treated dish (EIKEN CHEMICAL; AG2000).

This was cultured in an incubator (MCO-17AIC; manufactured by SANYO Electric Co., Ltd.) at 37° C. and 5% CO₂ for 36 hours.

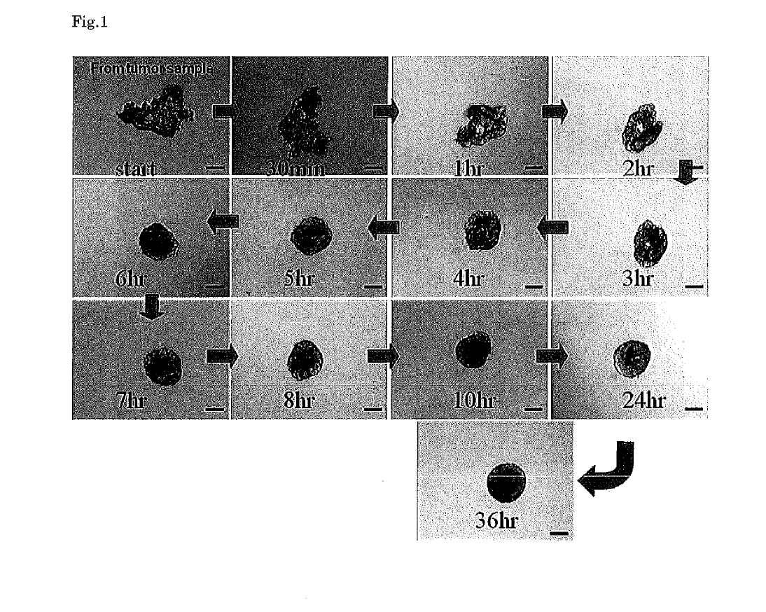

As a result, the cancer tissue-derived cell mass changed its irregular form into a regular sphere with the lapse of time as shown in FIG. 1, i.e., it became almost a sphere at least 3 to 6 hours later, and a completely regular sphere-shaped cell mass derived from the cancer tissue was obtained after 24 hours. As used herein, the cancer tissue-derived cell mass at a stage of culture being performed under such conditions refers to a cancer tissue-derived cell mass obtained under normal conditions.

### Example 2

### Preparation of Cancer Tissue-Derived Cell Mass from Surgical Specimens of Human Colon Cancer

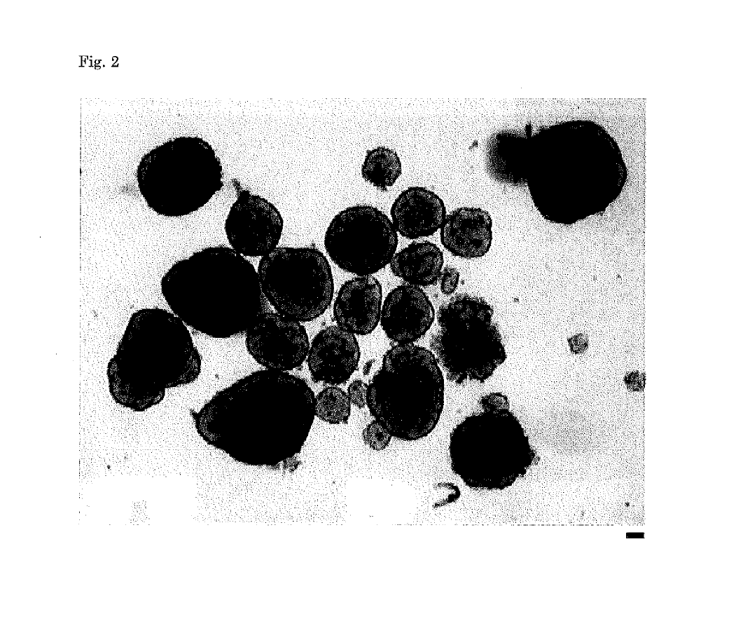

The cancer tissue-derived cell mass was obtained in the same manner as in Example 1, except that surgical specimens of colon cancer were used. As a result, a cancer tissue-derived cell mass of similar shape could be obtained from a different patient. That is, as shown in FIG. 2, an almost sphere-shaped cancer tissue-derived cell mass was obtained at least 12 hours later (C45, CB3, and C111).

### Example 3

### Culture of Cancer Tissue-Derived Cell Mass

The cancer tissue-derived cell mass obtained in Example 2 was purified as a composition and cultured in a dormancy medium (20 ml): DMEM/F12 with GlutaMAX (Invitrogen) 18.5 ml, 25% BSA (Invitrogen) 1.44 ml, and 2ME Stock 36.2 11.1 in a 6 cm non-treated dish (EIKEN CHEMICAL: AG2000) in a personal gas incubator (APM-30D, ASTEC Co., Ltd.) or INVIVO2 400 (RUSKINN) for 15 days at 37° C. under the conditions of an oxygen concentration of 1%.

Then, the culture medium was changed and culture was performed in a StemPro medium (100 ml): DMEM/F12 with GlutaMAX (Invitrogen) 90.8 ml, StemPro hESC SFM Supplement (Invitrogen) 2 ml, 25% BSA (Invitrogen) 7.2 ml, FGF-basic (10 μg/ml) 80 μl, and 2ME (55 mM) 182 l in a 6 cm non-treated dish (EIKEN CHEMICAL: AG2000) at 37° C. under a changed atmosphere (under normal conditions) of an oxygen concentration of 20%.

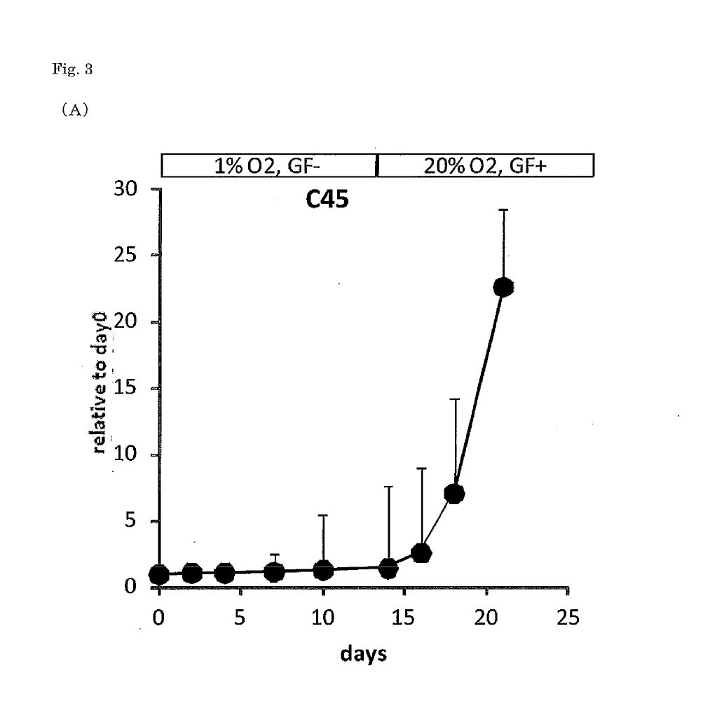

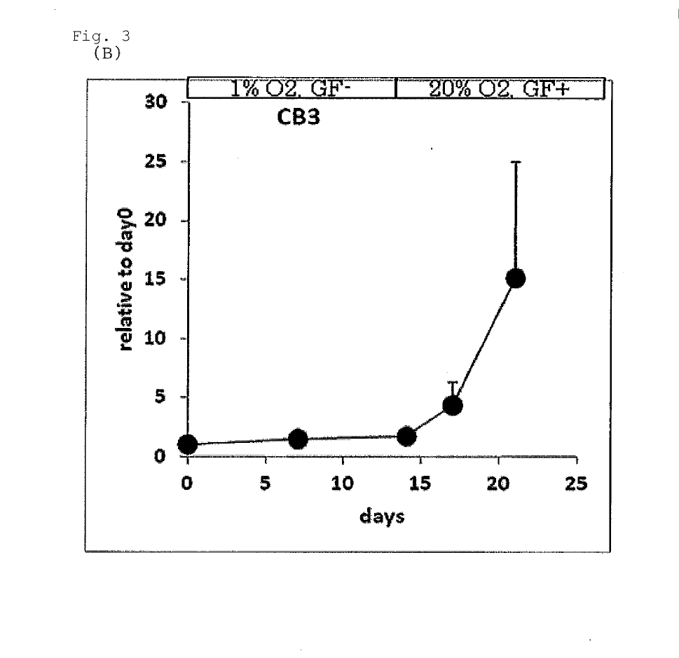

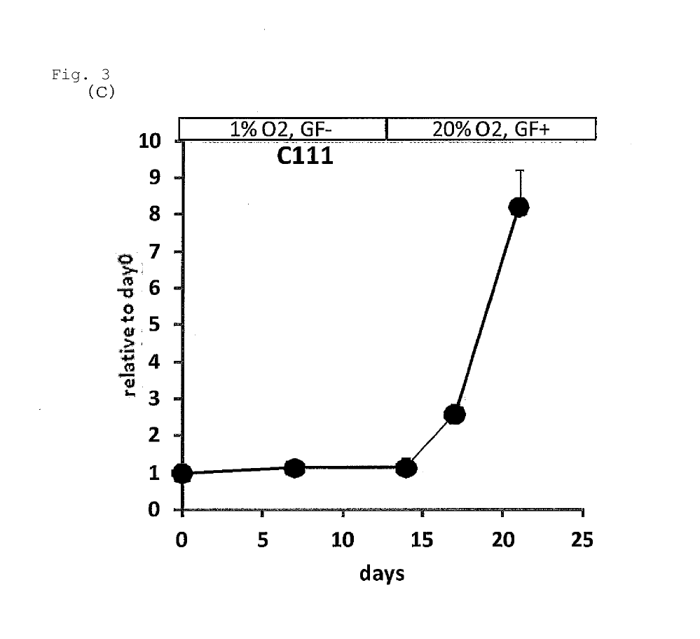

As a result, as shown in FIG. 3, proliferation of the cancer tissue-derived cell mass under the conditions of hypoxia and low levels of growth factors was suppressed in comparison with their normal growth conditions. In addition, when returned to normal culture conditions of 20% oxygen and supplemented growth factors, the cancer tissue-derived cell mass grew up rapidly.

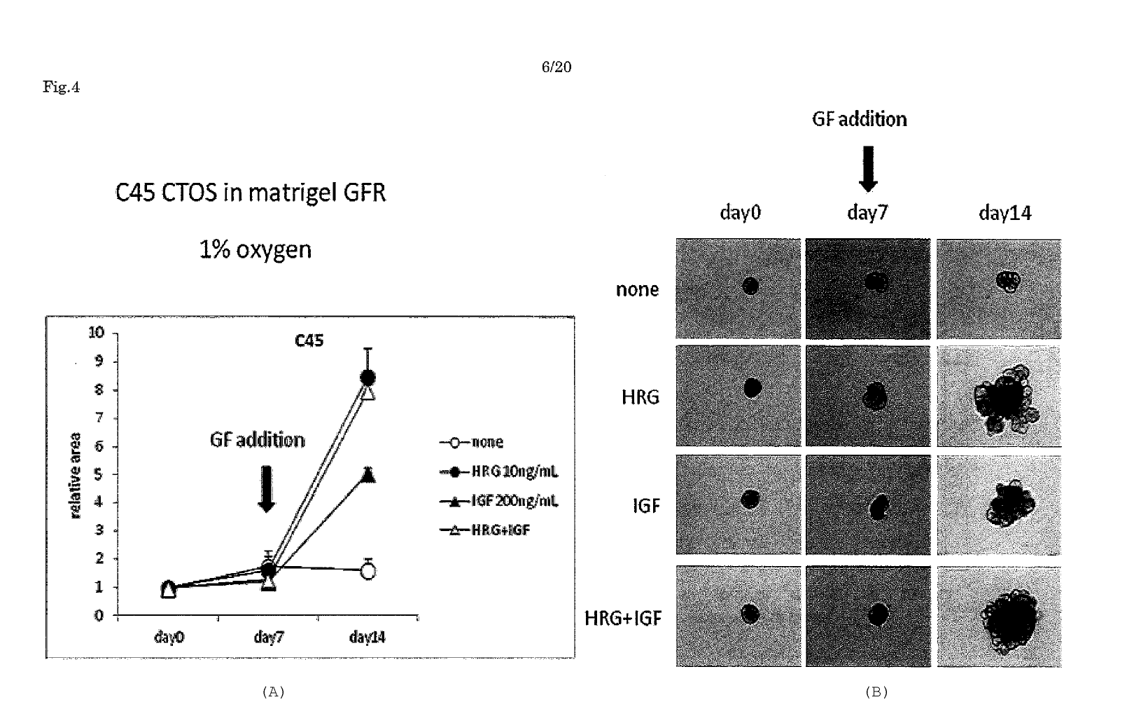

On the other hand, as shown in FIG. 4, when heregulin (10 ng/ml) or IGF (200 ng/ml), or both were added to a dormancy medium even in the hypoxic conditions, the cancer tissue-derived cell mass grew up rapidly.

In addition, under such culture conditions, oxygen consumption and glucose concentration in each medium were measured. The oxygen partial pressure and oxygen consumption were measured by a cellular respiration activity measurement apparatus (manufactured by Clino Ltd., CRAS-1.0). The glucose concentration was measured using Glucose CII-Test Wako (Wako Pure Chemical Industries, Ltd.). As a result, as shown in FIGS. 5(A) and 5(B), under the conditions of either or both of hypoxia and low levels of growth factors, it was found that the oxygen consumption and glucose consumption of the cancer tissue-derived cell mass were suppressed in comparison with the normal culture conditions. (In the Figure, □ shows “growth factor+20% O₂”, Δ shows “no growth factor+20% O₂”, ⋄ shows “no growth factor+1% O₂”, and ∇ shows “growth factor+1% O₂”.)

### Example 4

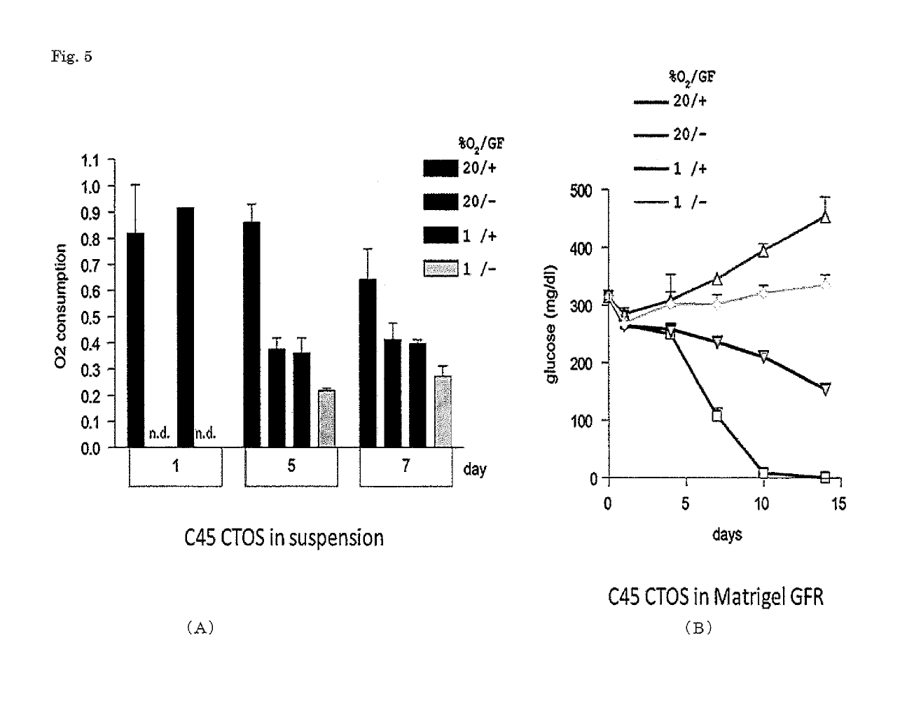

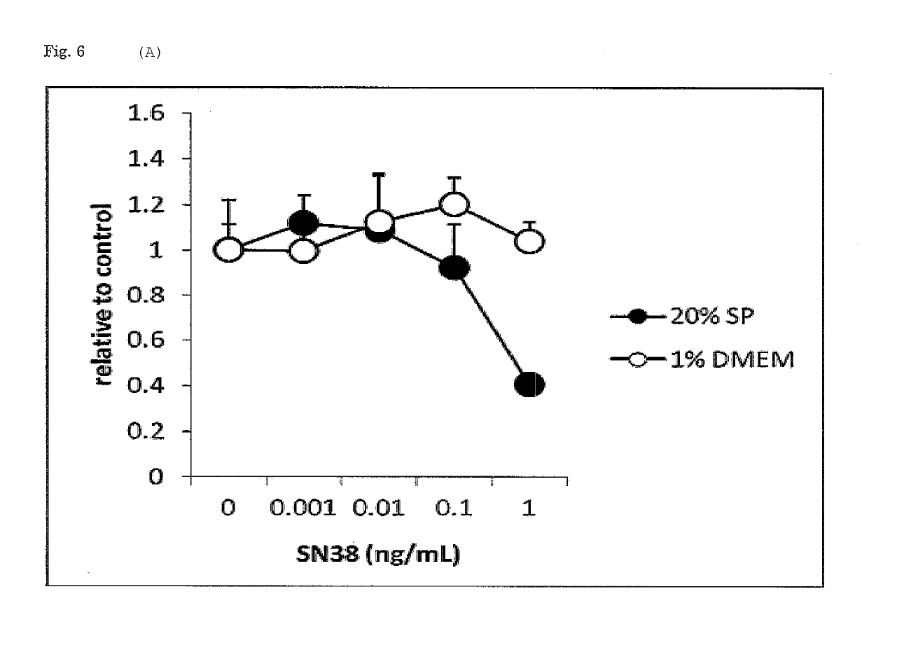

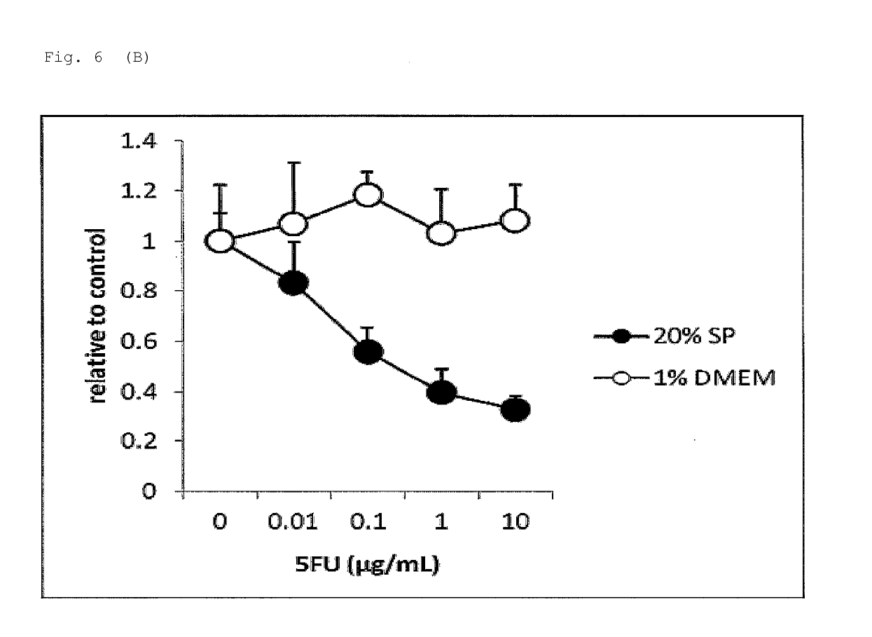

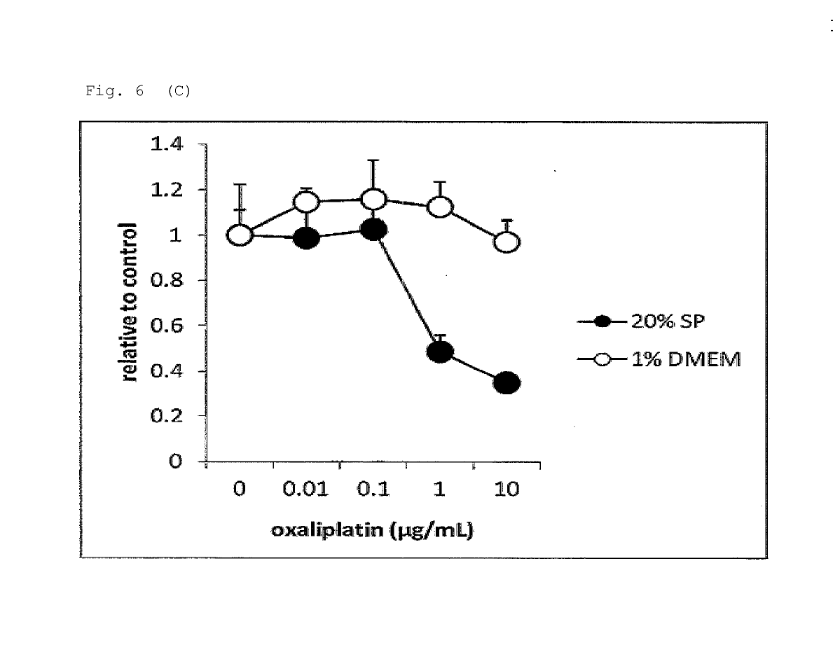

Using SN38,5-FU, and oxaliplatin known as anti-cancer agents, a drug sensitivity test was performed with C45 sample of Example 2. The test was proceeded by culturing the C45 obtained in example 2, in 1 cc of a dormancy medium or StemPro hESC SFM (Gibco; A10007-01) medium. Further, as to each drug, 5-FU and oxaliplatin were applied at concentrations of 0.01 μg/ml, 0.1 μg/ml, 1 μg/ml, and 10 μg/ml, and SN38 was applied at concentrations of 0.001 ng/ml, 0.01 ng/ml, 0.1 ng/ml, and 1 ng/ml, and comparative evaluation of the culture state was performed on day 7 after each culture. The results are shown in FIG. 6. An increase rate of the area of the cancer tissue-derived cell mass was relatively expressed when the increase rate of the area under culture with no drug application was expressed as 1. In FIG. 6, the proliferation of cancer cells in the cancer tissue-derived cell mass under normal culture conditions was suppressed on day 7 after culture, depending on the concentration of the drug, but the cancer tissue-derived cell mass in a dormant state did not respond to the concentration of drugs at all or responded little to it.

### Example 5

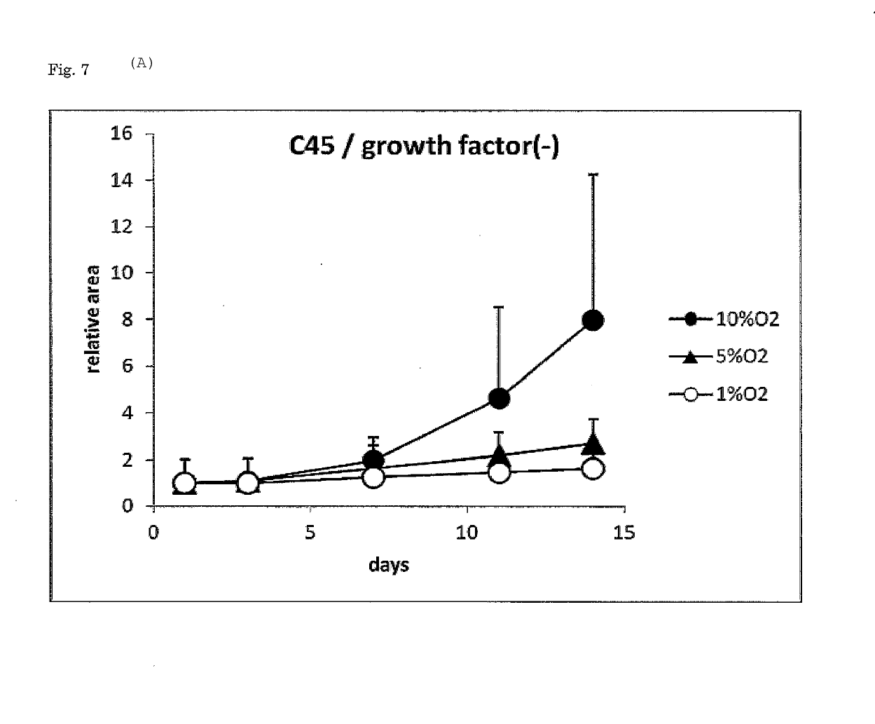

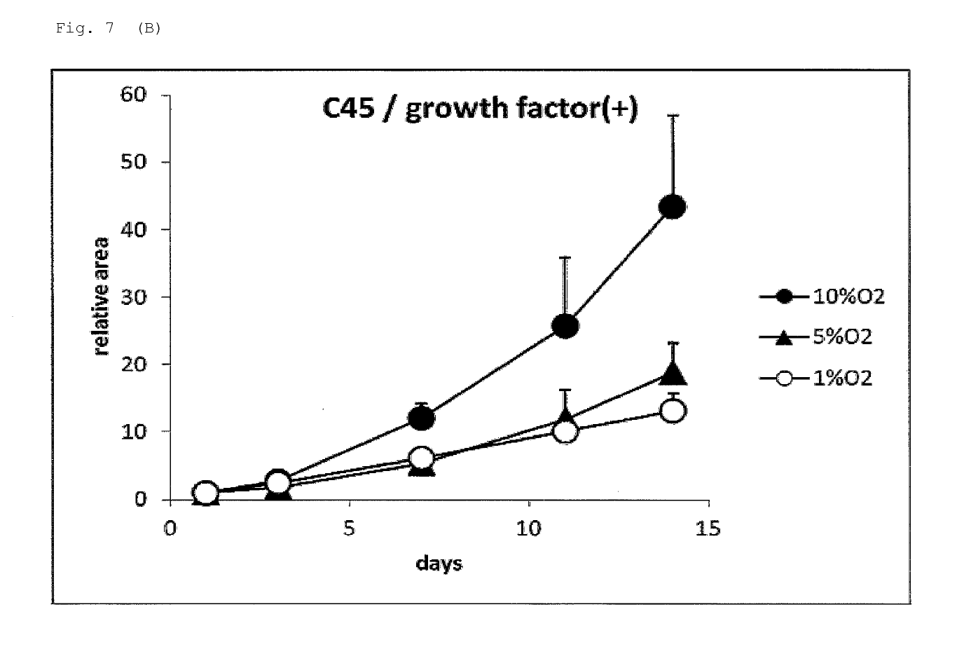

The relationship between the dormant state of the cancer tissue-derived cell mass and the partial oxygen pressure of culture environment were examined. The culture condition is a dormancy medium or a StemPro hESC SFM (Gibco; A10007-01) medium. As a result, as shown in FIG. 7, it was found that the growth was suppressed in 5% O₂ and 1% O₂. This trend was particularly evident under the conditions with no growth factors.

### Example 6

### Radiation Exposure Test

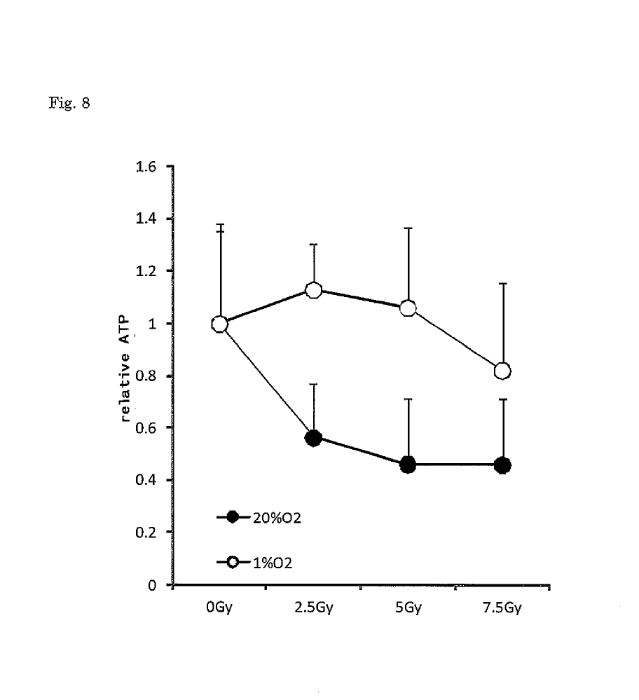

The cell masses derived from the cancer tissue obtained in the same manner as in Example 2 and used in the present invention, having a diameter of about 100 micrometers, were embedded in a collagen gel (CellMatrix type IA (Nitta Gelatin Inc.):5×DMEM (Gibco; 12100-038): buffer solution for gel reconstruction (50 mM NaOH, 260 mM NaHCO_{3, 200} mM HEPES)=7:2:1), and inoculated (×10 cell masses each) to 1 cc of STEMPRO serum-free medium (Gibco Company) for human ES cells in an incubator under the culture conditions of 37° C. and 5% CO₂ and then cultured. This was irradiated by γ-rays emitted from a cobalt isotope as a radiation source, thereby to detect the state of the cell mass by a relatively expressed area when an increase rate of the area under no radiation exposure is expressed as 1 and a relative ATP amount. Using a Celltiter-Glo (registered trademark) luminescent cell viability assay (Promega, G7570), the amount of cellular ATP was measured according to the manufacturer's instructions. Prior to the addition of the CellTiter-Glo reagent, Matrigel GFR was digested with a 0.2 mg/mL collagenase type 4 solution (Worthington, CLS4) to release a cancer tissue-derived cell mass. The results are shown in FIG. 8. In FIG. 8, the horizontal axis represents a radiation dose and the vertical axis represents a relative ATP amount corrected by the area before irradiation.

From the results, the tissue-derived cell mass in a dormant state of the present invention was found to be resistant to cobalt irradiation.

It should be noted that evaluation items, such as in the examples, were measured as below-described.

### Example 7

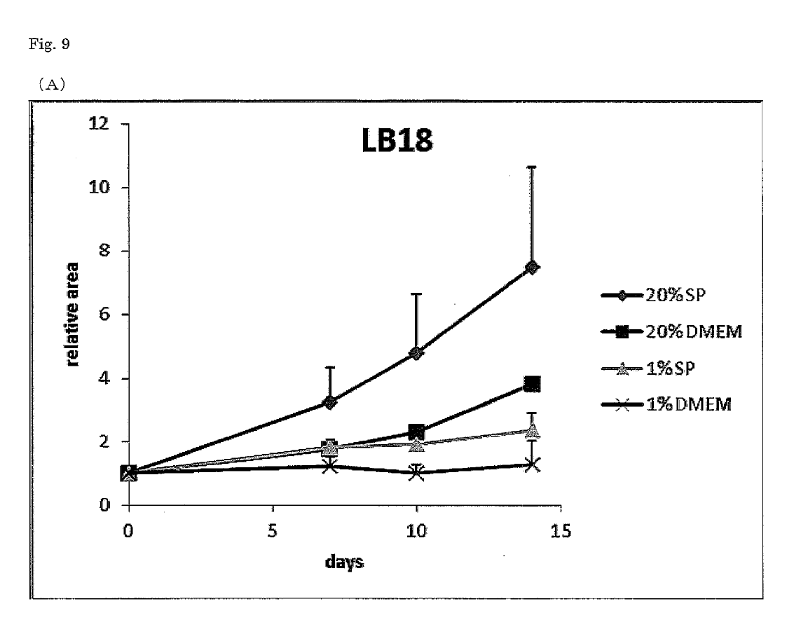

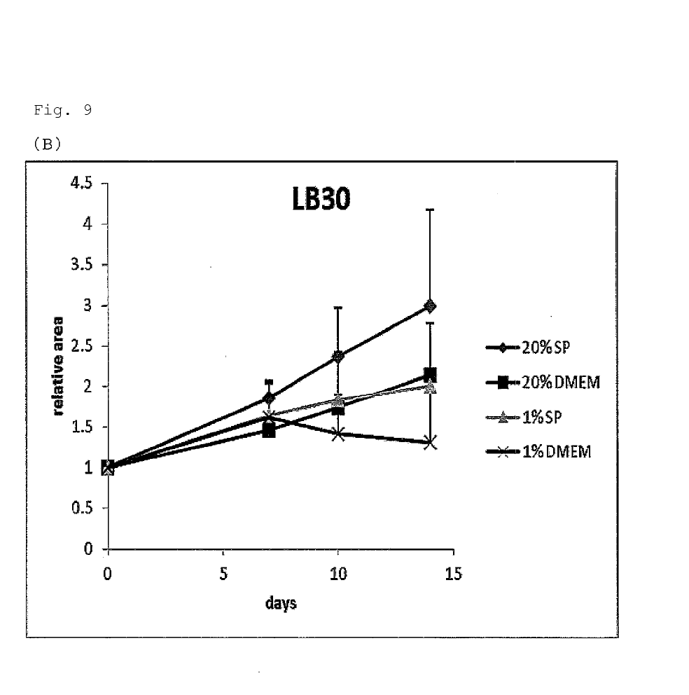

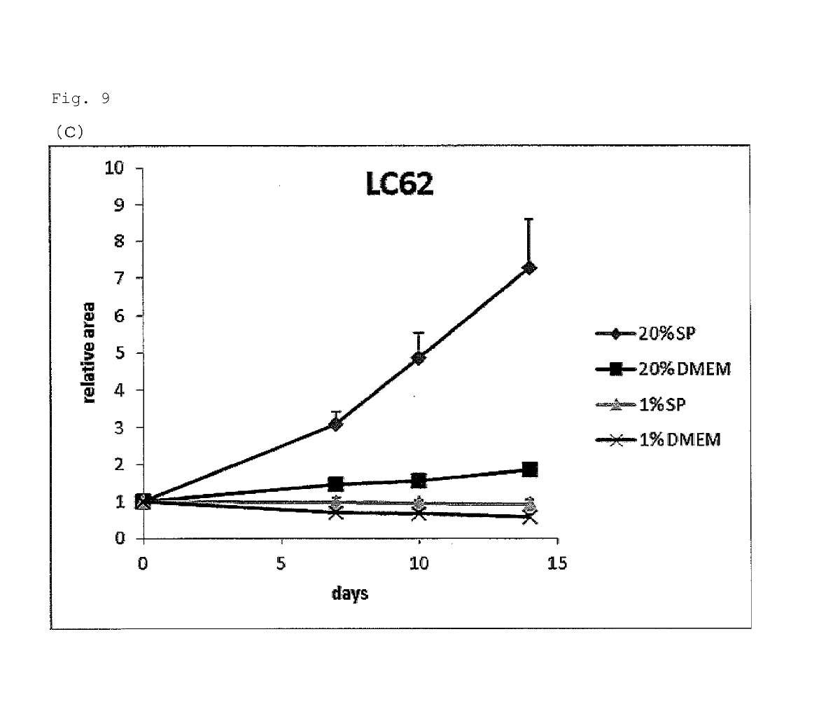

In the same manner as in Example 2, a cell mass derived from cancer tissue was prepared from lung cancers. After that, the relationship between the dormant state of the cancer tissue-derived cell mass, and the partial oxygen pressure of culture environment and the presence or absence of growth factors were examined. The culture condition is a dormancy medium or a StemPro hESC SFM medium. As a result, as shown in FIGS. 9A, 9B, and 9C, the dormant state was found to be reproduced under the state of a partial oxygen pressure of 1% and low levels of growth factors.

### Example 8

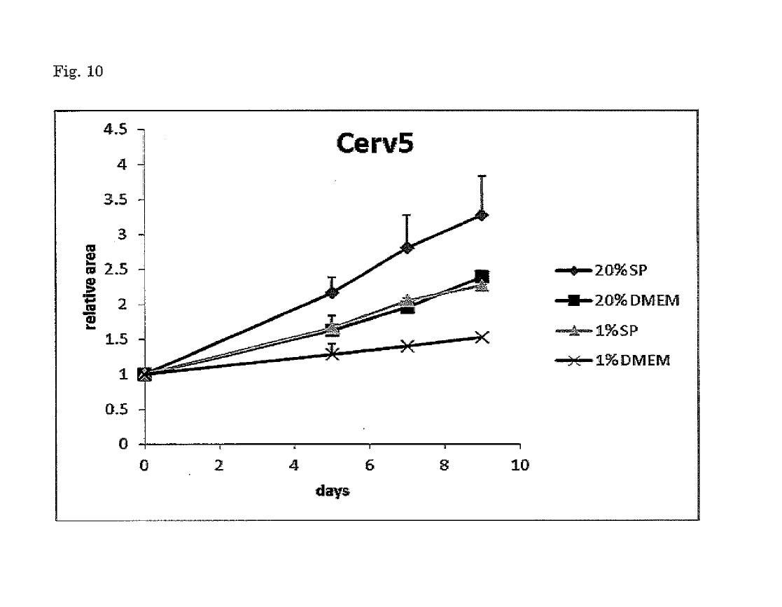

In the same manner as in Example 2, a cell mass derived from cancer tissue was prepared from uterine cancers. After that, the relationship between the dormant state of the cancer tissue-derived cell mass, and the partial oxygen pressure of culture environment and the presence or absence of growth factors were examined. The culture condition is a dormancy medium or a StemPro hESC SFM medium. As a result, as shown in FIG. 10, it was found that the dormant state was reproduced under the state of a partial oxygen pressure of 1% and low levels of growth factors.

### Example 9

The cancer tissue-derived cell mass obtained in Example 2 was obtained as a pure composition, which was then cultured for 15 days at 37° C. in a personal gas incubator (APM-30D, ASTEC Co., Ltd.) or INVIVO2 400 (RUSKINN) under the conditions of an oxygen concentration of 1% in a dormancy medium (20 ml) in a 6 cm non-treated dish (EIKEN CHEMICAL: AG2000).

Then, the culture was performed with or without change of the medium. That is, when the medium was changed, culture was performed in a StemPro medium (100 ml): DMEM/F12 with GlutaMAX (Invitrogen) 90.8 ml, StemPro hESC SFM Supplement (Invitrogen) 2 ml, 25% BSA (Invitrogen) 7.2 ml, HRG (10 μg/ml) 80 μl, and 2ME (55 mM) 182 μl in a 6 cm non-treated dish (EIKEN CHEMICAL: AG2000) at 37° C. without changing the oxygen concentration. SN38,5-FU, and oxaliplatin were respectively applied to cultured products at various concentrations.

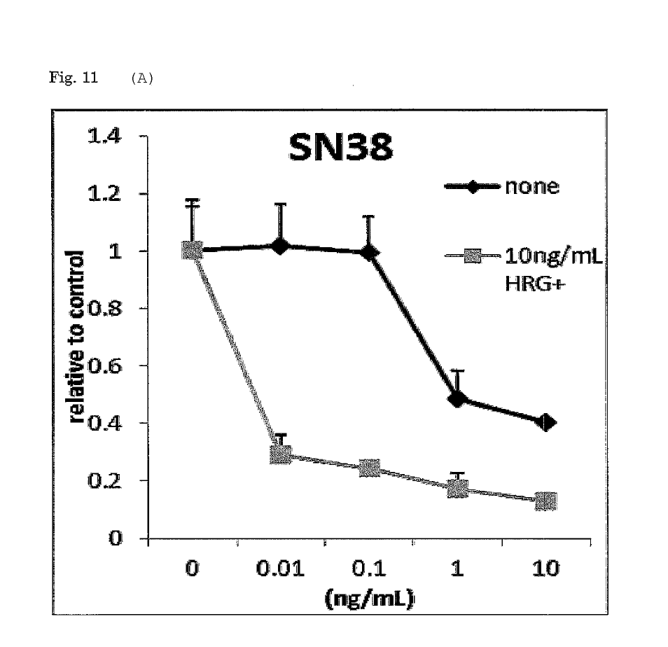

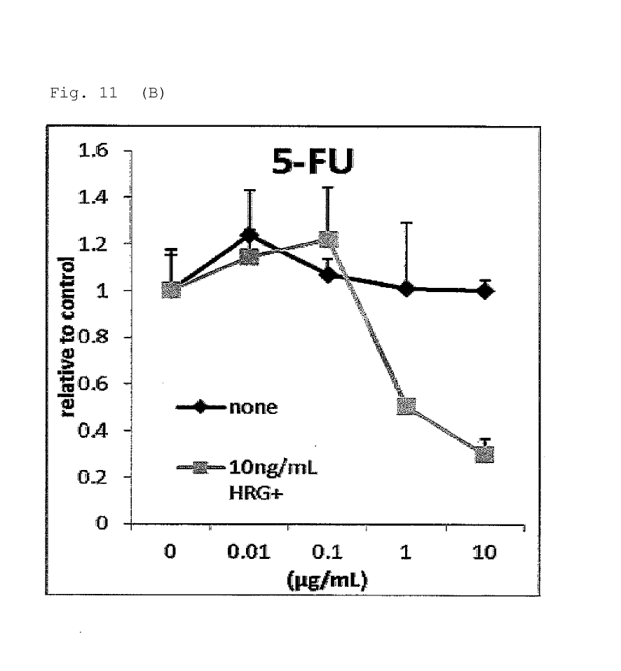

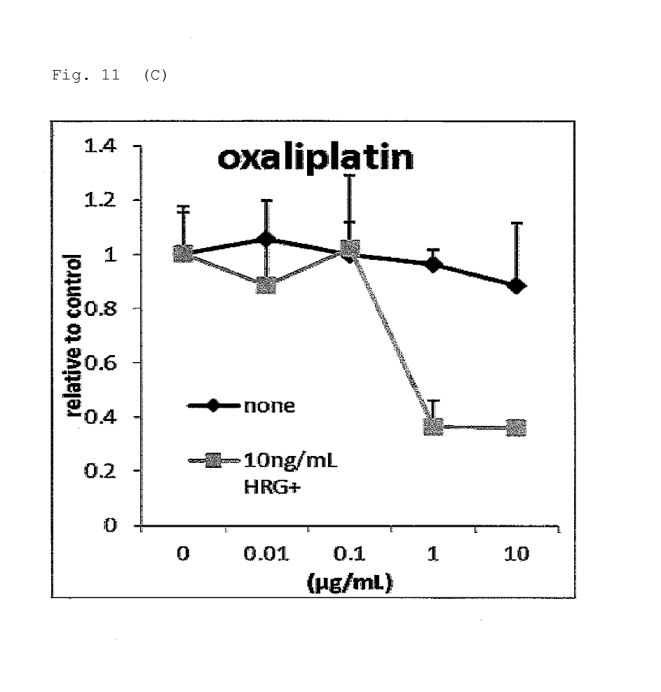

As a result, as shown in FIG. 11, when the medium was used without change, the effect on cell growth was small even after applying the drug, and when the medium was changed to contain a growth factor, the cell growth after applying the drug was affected. By using the cancer tissue-derived cell mass of the present invention, it is possible to reversibly return the dormant state to the original state, and it was confirmed that the effect of the drug was seen in such an original state.

### <Identification of Surface Antigen>

The cancer tissue-derived cell mass obtained in Example 1 was dispersed to single cells using trypsin/EDTA. These cells were reacted with a surface antigen-specific antibody that was labeled with a fluorescence substance, and then analyzed by a flow cytometry. As a result, existence of cells expressing a surface antigen uniformly at the same time was confirmed.

### <Confirmation of Basement Membrane-Like Material>

The cancer tissue-derived cell mass obtained in Example 1 was cultured for three days in 1 cc of STEMPRO serum-free medium (Gibco Company) for human ES cells in an incubator under the culture conditions of 37° C. and 5% CO₂. Antigenicity of laminin was observed in the cytoplasm of the cell in or near to the circumference of the cancer tissue-derived cell mass when this was fixed with formalin, embedded in paraffin, cut into thin slices, and anti-laminin antibody staining (rabbit anti-mouse laminin antibody; manufactured by Sigma-Aldrich Corporation) was performed according to the manufacturer's instructions. This revealed that laminin surrounded the circumference of a population of the cancer cells in the cancer tissue-derived cell mass according to the present invention. On the other hand, expression of laminin was not confirmed within 24 hours after treatment of surgical specimens.

### <Detection of Hypoxia>

### Example of Hypoxia Detection Using Pimonidazole

Pimonidazole that is a nitroimidazole compound has a characteristic to form an adduct with proteins or nucleic acids in the absence of oxygen. The hypoxic region of the tissue treated with pimonidazole under hypoxic conditions can be recognized using an antibody that specifically recognizes pimonidazole. There is a hypoxic region in the cancer tissue separated by about 100 micrometers from a blood vessel. A hypoxic region appears, and a wide range of cell death was observed inside (hypoxic region) the boundary apart from about 100 micrometers from the circumference of the cancer tissue-derived cell mass obtained in Example 1. Therefore, when the state of cell death is evaluated in relation to drugs in the present specification, it is preferred to evaluate it in comparison with the cancer tissue-derived cell mass cultured under normal conditions.

### <Evaluation of In Vitro Proliferation Ability>

The in vitro proliferation ability of the cancer tissue-derived cell mass was examined as follows. The cancer tissue-derived cell masses (×10 cell masses each) obtained in example were embedded in a collagen gel (CellMatrix type IA (Nitta Gelatin Inc.): 5×DMEM (Gibco; 12100-038): buffer solution for gel reconstruction (50 mM NaOH, 260 mM NaHCO_{3, 200} mM HEPES)=7:2:1), and was cultured in 1 cc of STEMPRO serum-free medium (Gibco Company) for human ES cells in an incubator under the culture conditions of 37° C. and 5% CO₂. The cell state was observed periodically and the size of the cell was measured with a phase contrast microscope (magnification 40 times) equipped with a CCD camera.

### <Confirmation of Cell Count>

A 100 to 250 μm-sized cancer tissue-derived cell mass was treated with trypsin 0.25% and EDTA 2.6 mM for three minutes in the same manner as in Example 1, and mechanically degraded by pipetting approximately 30 times. This was diluted and subdivided into a 96-well culture plate so that one cell can be placed in one well. The cell count constituting a cell mass that was non-single celled was counted and recorded. Then, culture (under the conditions as above) was performed to record an increase of the cell count of each well, and the culture was observed for 30 days.

### <Evaluation of Presence or Absence of In Vitro Cell Death>

The situation of survival of the cancer tissue-derived cell mass was examined by Propidium Iodide (PI) staining method. PI was added to the medium to a final concentration of 1 μg/ml and culture was performed for 10 minutes. The presence or absence of in vitro cell death was evaluated by a microscopic observation.

### <Transplantation Test in Different Species of Animals>

The cancer tissue-derived cell masses (×10) having each a diameter of about 100 μm obtained in Example 2 by culture for three days according to the present invention were suspended in Matrigel (BD Corporation), and the suspension was administered subcutaneously to the back of NOD-SCID mice for transplantation. The evaluation of tumorigenesis was performed by measuring the size of the tumor with the lapse of time. As a result, it was confirmed that a marked tumorigenesis was seen in an individual of mice which had been transplanted with the cancer tissue-derived cell mass in a dormant state of the present invention, and the cancer tissue-derived cell mass according to the present invention has a high tumorigenic ability. When this tissue was analyzed, it was revealed that a similar tissue type was produced in both of the tumor occurred in transplanted mice and the existing tumor in a living body.

### <Evaluation of In Vitro Cell State>

The dormant state of the cancer tissue-derived cell mass of the present invention could also be detected by examining the state of phosphorylation of S6. That is, it was found that the phosphorylation of pS6 in a dormant state almost cannot be detected by immunostaining using an antibody against phosphorylation of S6. Furthermore, by the detection of the cell proliferation with a BrdU kit, it was revealed that uptake of BrdU in a dormant state was extremely low.

In accordance with the present invention, it was demonstrated that a dormant state of cancers could be accurately reproduced in vitro by using a cancer tissue-derived cell mass. The findings obtained in the present invention can be applied in vitro to a wide range of applications. In particular, the cancer tissue-derived cell mass of the present invention can be widely used in a drug sensitivity test using such a dormant state. In a method for evaluating a drug using the cancer tissue-derived cell mass of the present invention, it is also possible to obtain a drug different from the existing drug that targets cancer cells in the growth phase. That is, it may be possible to obtain a drug to prevent post-treatment relapse that has become a problem in cancer therapy.

1. A method of processing a cancer tissue-derived cell ass, comprising the step of
culturing in vitro the cancer tissue-derived cell mass from a patient under the conditions of hypoxia and low levels of growth factors,
wherein the cancer tissue-derived cell mass is a substantially spherical shape or spheroidal form and does not substantially contain cells other than cancer cells.

2. A method for evaluating the effect of a treatment for a cancer tissue-derived cell mass comprising the step of
applying the treatment to the cancer tissue-derived cell mass in a dormant state in vitro with no substantial growth and no substantial cell death and with reduction in glucose consumption and oxygen consumption, of which the proliferation ability is inherently retained,
wherein the cancer tissue-derived cell mass is a substantially spherical shape or spheroidal form and does not contain substantially cells other than cancer cells.

3. The method for evaluating the effect of a treatment for a cancer tissue-derived cell mass according to claim 2, wherein said treatment is selected from the group consisting of drug administration, radiation exposure, thermotherapy, immunotherapy, photochemotherapy, and gene introduction.

4. The method for evaluating ding to claim 2, comprising the step of
detecting proliferation state of the cancer tissue-derived cell mass, determining life and death of the cancer tissue-derived cell mass, or detecting change of intracellular signal transduction of the cancer tissue-derived cell mass.

5. A method for evaluating the effect of a treatment for a cancer tissue-derived cell mass, comprising the step of
culturing in vitro the cancer tissue-derived cell mass from a patient, while applying various treatments, and then culturing the cancer tissue-derived cell mass under the conditions of hypoxia and low levels of growth factors,
wherein the cancer tissue-derived cell mass is a substantially spherical shape or spheroidal form and does not contain substantially cells other than cancer cells.

6. The method for evaluating the effect of a treatment for a cancer tissue-derived cell mass according to claim 5, wherein said treatment is selected from the group consisting of drug administration, radiation exposure, thermotherapy, immunotherapy, photochemotherapy, and gene introduction.

7. The method for evaluating according to claim 5, comprising the step of
detecting proliferation state of the cancer tissue-derived cell mass, determining life and death of the cancer tissue-derived cell mass, or detecting change of intracellular signal transduction of the cancer tissue-derived cell mass.

8. A method for evaluating the effect of a treatment for a cancer tissue-derived cell mass from a patient, comprising the steps of
culturing in vitro the cancer tissue-derived cell mass from a patient under the conditions of a hypoxia and of low levels of growth factors, a step of applying a treatment to the cancer tissue-derived cell mass, and
culturing the cancer tissue-derived cell mass under the condition of at least 1 ng/ml to 200 ng/ml of a growth factor,
wherein the cancer tissue-derived cell mass is a substantially spherical shape or spheroidal form and does not contain substantially cells other than cancer cells.

9. The method for evaluating the effect of a treatment for a cancer tissue-derived cell mass according to claim 8, wherein the treatment is selected from the group consisting of drug administration, radiation exposure, thermotherapy, immunotherapy, photochemotherapy, and gene introduction.

10. The method for evaluating according to claim 8, comprising the step of
detecting proliferation state of the cancer tissue-derived cell mass, determining life and death of the cancer tissue-derived cell mass, or detecting change of intracellular signal transduction of the cancer tissue-derived cell mass.

11. The method for evaluating according to claim 2, comprising the step of
obtaining genetic information by evaluating the gene of the cancer tissue-derived cell mass in advance and correlating the genetic information with the treatment.

12. A method for screening a drug, comprising the step of
treating a cancer tissue-derived cell mass in a dormant state with a drug candidate compound in vitro, which cancer tissue-derived cell mass in a dormant state is with no substantial growth and no substantial cell death and with reduction in glucose consumption and oxygen consumption, of which proliferation ability is inherently retained,
wherein the cancer tissue-derived cell mass is a substantially spherical shape or spheroidal form and does not contain substantially cells other than cancer cells.

13. The method for screening a drug according to claim 12, comprising the step of
detecting proliferation state of the cancer tissue-derived cell mass, determining life and death of the cancer tissue-derived cell mass, or detecting change of intracellular signal transduction of the cancer tissue-derived cell mass.

14. A method for screening a drug to the cancer tissue-derived cell mass, comprising the step of
culturing in vitro a cancer tissue-derived cell mass from a patient in the presence of a drug candidate compound, and then culturing it under the conditions of hypoxa and low levels of growth factors,
wherein the cancer tissue-derived cell mass is a substantially spherical shape or spheroidal form and does not contain substantially cells other than cancer cells.

15. The method for screening a drug according to claim 14, comprising the step of
detecting proliferation state of the cancer tissue-derived cell mass, determining life and death of the cancer tissue-derived cell mass, or detecting change of intracellular signal transduction of the cancer tissue-derived cell mass.

16. A method for screening a drug to the cancer tissue-derived cell mass, comprising the steps of
culturing in vitro a cancer tissue-derived cell mass from a patient under the conditions of hypoxia and low levels of growth factors,
applying a drug candidate compound to the cancer tissue-derived cell mass, and
culturing the cancer tissue-derived cell mass under the condition of at least 1 ng/ml to 200 ng/ml of a growth factor,
wherein the cancer tissue-derived cell mass is a substantially spherical shape or spheroidal form and does not contain substantially cells other than cancer cells,

17. The method for screening a drug according to claim 16, comprising the step of
detecting proliferation state of the cancer tissue-derived cell mass, determining life and death of the cancer tissue-derived cell mass, or detecting change of intracellular signal transduction of the cancer tissue-derived cell mass.

18. The method for screening a drug according to claim 12, comprising the steps of
obtaining genetic information by evaluating the gene of a cancer tissue-derived cell mass in advance and
correlating the genetic information with the drug candidate compound.

19. A drug obtained by the method according to claim 12.

20. A cancer tissue-derived cell mass in a dormant state in vitro, which maintains such state for at least three days that the cancer-tissue derived cell mass does not substantially grow, does not substantially cause cell death, in a state of a glucose consumption of 10% or less and an oxygen consumption of 30% or less, in comparison with a normal state, and can be present in a state able to recover the proliferation ability, the glucose consumption and the oxygen consumption by culturing afterward under the conditions of at least 1 ng/ml to 200 ng/ml of a growth factor, the cancer tissue-derived cell mass being a substantially spherical shape or spheroidal form and does not contain substantially cells other than cancer cells.

21. The cancer tissue-derived cell mass according to claim 20, wherein the dormant state is a state where phosphorylation of Akt and/or S6 is reduced, and/or the amount of c-Myc protein is decreased to 10% or less compared to the normal state.
